(19) United States
(12) Patent Application Publication
(10) Pub. No.: US 2010/0184660 A1
(43) Pub. Date: Jul. 22, 2010

(54) SINGLE EXON GENES ENCODING FOR NOVEL BIO-ACTIVE PEPTIDES

(75) Inventors: Eva Jung, Bad Soden (DE); Werner Dittrich, Frankfurt (DE); Sabine Scheidler, Bad Soden (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/519,978

(22) PCT Filed: Dec. 12, 2007
(86) PCT No.: PCT/EP2007/010842

§ 371 Date: Feb. 24, 2010

(30) Foreign Application Priority Data
Dec. 19, 2006 (DE) 10 2006 059 825.3

(51) Int. Cl. A61K 38/16; C07K 14/00; C07H 21/04; C12N 15/74; C07K 1/14; C07K 1/00; C07K 1/02; C07K 16/00; C12N 1/21; C12P 21/00; A61P 3/10; A61P 9/12

(52) U.S. Cl. 514/12; 530/324; 530/350; 536/23.1; 435/320.1; 530/412; 530/333; 530/338; 530/339; 530/387.1; 435/252.3; 435/69.1

(57) ABSTRACT

The present invention relates to novel bio-active peptide hormone, process for the production of the same, and use of the same. The present invention identified novel bioactive peptide precursor and salts thereof which can be used as drugs, for example therapeutic polypeptides, ligands to discover relevant targets (e.g. GPCRs) or targets for drug intervention.

The present invention relates to novel bio-active peptide hormone, process for the production of the same, and use of the same. The present invention identified novel bioactive peptide precursor and salts thereof which can be used as drugs, for example therapeutic polypeptides, ligands to discover relevant targets (e.g. GPCRs) or targets for drug intervention.

The present invention relates to novel bio-active peptide hormone, a process for the production of the same, and use of the same. More particularly, the present invention relates to a method for the identification of bioactive peptide hormones derived from precursor proteins that can be used as therapeutic polypeptides, targets for drug intervention, ligands to discover relevant targets (e.g. GPCR deorphaning) or biomarkers to monitor diseases.

Many biologically active peptides exhibit profound effects both in health and disease, either by growth stimulation, growth inhibition, or the regulation of critical metabolic pathways.

Peptide hormones are produced as precursors in different cell types and organs like glands, neurons, intestine, brain; etc. Peptide hormones are initially synthesized as larger precursors, or prohormones, and may acquire a number of post-translational modifications during transportation through the ER and Golgi stacks. They are processed and transported to their final destination to act as active substances (first messengers) to trigger a cellular response by binding to a cell surface receptor. They play a key role in physiological processes that are relevant to many areas of biomedical research such as Diabetes (Insulin), blood pressure regulation (Angiotensin), Anemia (Erythropoietin-α), Multiple Sclerosis (Interferon-β) and others.

The recent sequencing of the human genome has revealed around 30,000 genes, far fewer than would be predicted from the complexity of human biological processes. Alternative splicing of these genes prior to translation would likely generate up to 200,000 primary transcripts. It is now widely accepted that post translational modifications to the protein products coded by these genes represents the additional level of complexity required, to explain the diversity of function.

Gene finding typically refers to the area of computational biology that is concerned with algorithmically identifying stretches of sequence, usually genomic DNA, that are biologically functional. This especially includes protein-coding genes, but may also include other functional elements such as RNA genes and regulatory regions. Gene finding is one of the first and most important steps in understanding the genome of a species once it has been sequenced.

In extrinsic gene finding systems, the target genome is searched for sequences that are similar to extrinsic evidence in the form of the known sequence of a messenger RNA (mRNA) or protein product. Given an mRNA sequence, it is critical to derive a unique genomic DNA sequence from which it had to have been transcribed. Given a protein sequence, a family of possible coding DNA sequences can be derived by reverse translation of the genetic code. Once candidate DNA sequences have been determined, it is an algorithmic problem to efficiently search a target genome for matches, complete or partial, and exact or inexact. BLAST is a widely used system designed for this purpose.

A high degree of similarity to a known messenger RNA or protein product is strong evidence in many cases that a region of a target genome is a protein-coding gene. However, to apply this approach systemically requires extensive sequencing of mRNA and protein products. Not only is this expensive, but in complex organisms, only a subset of all genes in the organism's genome are expressed at any given time, meaning that extrinsic evidence for many genes is not readily accessible in any single cell culture. Thus, in order to collect extrinsic evidence for most or all of the genes in a complex organism, many hundreds or thousands of different cell types must be studied, which itself presents further difficulties. For example, some human genes may be expressed only during development as an embryo or fetus. Despite these difficulties, extensive transcript and protein sequence databases have been generated for human as well as other important model organisms in biology, such as mice and yeast. For example, the RefSeq database contains transcript and protein sequence from many different species and the Ensembl system comprehensively maps this evidence to human and several other genomes.

Because of the inherent expense and difficulty in obtaining extrinsic evidence for many genes, it is also necessary to resort to ab initio gene finding, in which genomic DNA sequence alone is systematically searched for certain telltale signs of protein-coding genes. These signs can be broadly categorized as either signals, specific sequences that indicate the presence of a gene nearby, or content, statistical properties of protein-coding sequence itself. Ab initio gene finding might be more accurately characterized as gene prediction, since extrinsic evidence is generally required to conclusively establish that a putative gene is functional.

Ab initio gene finding in eukaryotes, especially complex organisms like humans, is considerably more challenging for several reasons. First, the promoter and other regulatory signals in these genomes are more complex and less well-understood than in prokaryotes, making them more difficult to reliably recognize. Two classic examples of signals identified by eukaryotic gene finders are CpG islands and binding sites for a poly (A) tail.

Second, splicing mechanisms employed by eukaryotic cells mean that a particular protein-coding sequence in the genome is divided into several parts (exons), separated by non-coding sequences (introns). Splice sites are themselves another signal that eukaryotic gene finders are often designed to identify. A typical protein-coding gene in human might be divided into a dozen exons, each less than two hundred base pairs in length, and some as short as twenty to thirty. It is therefore much more difficult to detect periodicities and other known content properties of protein-coding DNA in eukaryotes.

Advanced gene finders for both prokaryotic and eukaryotic genomes typically use complex probabilistic models, such as hidden Markov Models, in order to combine information from a variety of different signal and content measurements. The Glimmer system is a widely used and highly accurate gene finder for prokaryotes. Eukaryotic ab initio gene finders, by comparison, have achieved only limited success; a notable example is the GENSCAN program.

The present invention identified novel bioactive peptide hormone precursors by means of identification of novel single exon genes that encode for peptide hormone precursor sequences. To find novel single exon genes, the human genome (NCBI 33 assembly, 1 Jul. 2003) and the mouse genome (NCBI 30 assembly, 1 Jul. 2003), both were translated into all six reading frames using the standard genetic code. Only sequence fragments starting with the amino acid Methionine and with a length between 50 and 200 amino acids were selected. The human and the mouse set were compared to each other using the program BLAST in order to find closely related sequences in both organisms. Only sequences that appear in both organisms (human and mouse) were selected. To filter for secreted proteins, potential signal sequences were predicted with the program signalP and the absence of potential membrane spanning regions was confirmed by the program TMHMM. In addition, an InterPro search was performed on the selected sequences in order to rule out the presence of readily described protein domains (e.g. kinase domain etc). The novelty of the remaining sequences was verified by sequence comparison to publicly available databases such as UNIPROT. These in silico analysis suggested the discovery of novel secreted proteins that lack any previously described protein domains.

It is generally understood, that peptide hormones are characterized by their high specificity as well as their effectiveness in very low concentrations. Another characteristic of peptide hormones is that their corresponding mRNA is expressed in a small number of distinct tissues. A ubiquitous expression pattern of peptide hormones is rarely observed in mammalian systems.

To determine the tissues transcribing the 8 novel genes in the human body, commonly used in vitro transcription assays were performed on a panel of human tissues (see FIG. 1-6). Using specific probe/primer sets, the mRNA encoding the gene of interest can be detected and quantified. However, it must be noted that the gene expression data can be influenced by the transcription of genes located in the same locus on the genome. In addition the tissue panel used in the present invention is not comprehensive.

Bioactive peptides hormones have a tremendous use in biomedical research and are therefore of interest to the pharmaceutical industry. Various peptide hormones are used for treatment of diseases.

Whereas WO2004039956 (Title: “compositions and methods for treatment of immune related diseases”) disclose several bioactive polypeptide sequences, the method of identification and the use of such sequences had been made however no reference to.

The present invention identified novel genes encoding for bioactive peptide hormone precursors. Peptide hormones are characterized by their high specificity as well as their effectiveness in very low concentrations. Bioactive peptides hormones have a tremendous use in biomedical research. Various peptide hormones are used for treatment of diseases or to monitor a disease states. Such polypeptide sequences can be used in therapeutically effective amounts as medicines and medicaments for immune related diseases.

The problem as laid out by availability of the closest prior art (WO2004039956) could be defined as to identify novel hormone polypeptide sequences which are useful for the treatment of human diseases. The present invention solves that problem by providing 8 novel peptide hormone precursors and fragments thereof which could be used to interfere with physiological factors that increase the risk of arteriosclerosis, inflammation or uncontrolled cell division. According to the invention it will provide a new reservoir of hormonal polypeptides that could be used for treatment of human disease in the field of biomedical research.

The present invention therefore refers to polypeptide chain consisting of an amino acid sequence according Seq id no 1-8 or an amino acid sequence derived there from by deletion, substitution or insertion of at least one amino acid residue, its amide or ester or a salt of said peptide.

An embodiment of present invention refers to a polypeptide chain consisting of the following amino acid sequence:

MYWMALRRISTLGSRWLGLSRVLLFRASKASFTFLSLRFSLSVAARRRST
DTDFLLHTLHAHGRHWPGQCSGVPSPLSSRGPGASGLRVSSVRS

Another embodiment of present invention relates to a polypeptide consisting of following amino acid sequence:

MGSGCARARLGLLSWLAASSGSEDALASSISVKLALELAEVAWSEGDEAE
GLAPWLSPLVQGRDSGEDREQLEAACLKRGSWAGAGKARELSPTAPKWLE
EAEERLTLRSIPL

Another embodiment of present invention relates to a polypeptide consisting of the following amino acid sequence:

MLLAMSSISIFSSLFSFSSFCFTRCRLSICSPSSATLSACFFLRVAAVAS
CCRVASSRSLRIFWNSASLFLFISIWAEVAPLASSSLSLISSSSLARSER
CFSILALSVCSASISSSSSSMRA

Another embodiment of present invention relates to a polypeptide consisting of the following amino acid sequence:

MATSWAGSAAPPASAAKSVVGTRPSRPGGPRSAWRRRRATLAAWTGPARA
ATATTTRAAARRPVAARTPARLAATSRATHARTWPMASPRASVTTCTCAF
RAARASPALSS

Another embodiment of present invention relates to a polypeptide consisting of the following amino acid sequence:

MPRSAPRAAAAPARAPAAAAVACACCPNSAPDFFMVCGGHVRSLAGKRLF
SSPPRPACSGPNDLRSSGVSGGAVRPAARTRRRAQGEVEEEASCGEKGRR
TAERMGPVAAARAGLDAAWARRCEVPKVTTIPTRQPRAPARPGAPRRI

Another embodiment of present invention relates to a polypeptide consisting of the following amino acid sequence:

MPKWRLAWPKQTRASSCGLSLPSISCASSCSASRNGGDRCSLRTTTTRHT
R

Another embodiment of present invention relates to a polypeptide consisting of the following amino acid sequence:

MSVWTFLKCRGNSSLLKNLLQVKVKAELLLLCLLVTHSLWSSTWSPPGVA
AVRSASTVPEENCSGSKLYVCVAKSMNSPSMLLDSEMTWPLSSLSKAHWR
WLMRSDLGRSSTVIPKSEVSTALCSLGLQLNMASPSRARFPQ

Another embodiment of present invention relates to a polypeptide consisting of the following amino acid sequence:

MASAAGEPFSMYLASAAAALCTPTASARKARGLRTEPLDEVLARGGPAAS
TLWCRCRLWPKASLYPGARKPCLAASGSDSSTSGGSATDTGPDLTPWKEV
DSDLSASMQLLMIWLTLSTSLAMVEISATELWLSGPGRPSSQSLRSGGSP
VRTSM

An embodiment of the invention also provides a DNA comprising of a nucleotide base sequence according to Seq Id no 9 to 16 encoding a polypeptide chain comprising an amino acid sequences represented by Seq Id no 1-8 or its amide, ester or a salts thereof.

This invention relates to a DNA that consist of a nucleotide base sequence according to Seq Id no 9, encoding a polypeptide chain that consist of an amino acid sequence according to Seq Id no 1 or its amide, ester or a salts thereof.

The Present invention relates to a DNA that consists of a nucleotide base sequence according to Seq Id no 10, encoding polypeptide chain described in paragraph 1 that consists of an amino acid sequence according to Seq Id no 2 or its amide, ester or a salts thereof.

Another embodiment of present invention relates to a DNA that consist of a nucleotide base sequence according to Seq Id no 11, encoding polypeptide chain that consist of an amino acid sequence according to Seq Id no 3 or its amide, ester or a salts thereof.

Another embodiment of present invention relates to a DNA that consist of a nucleotide base sequence according to Seq Id no 12, encoding polypeptide chain that consist of an amino acid sequence according to Seq id no 4 or its amide, ester or a salts thereof.

Another embodiment of present invention relates to a DNA that consist of a nucleotide base sequence according to Seq Id no 13, encoding polypeptide chain that consist of an amino acid sequence according to Seq Id no 5 or its amide, ester or a salts thereof.

Another embodiment of present invention relates to a DNA that consist of a nucleotide base sequence according to Seq Id no 14, encoding polypeptide chain that consist of an amino acid sequence according to Seq Id no 6 or its amide, ester or a salts thereof.

Another embodiment of present invention relates to a DNA that consist of a nucleotide base sequence according to Seq Id no 15, encoding polypeptide chain that consist of an amino acid sequence according to Seq Id no 7 or its amide, ester or a salts thereof.

Another embodiment of present invention relates to a DNA that consist of a nucleotide base sequence according to Seq Id no 16, encoding polypeptide chain that consist of an amino acid sequence according to Seq Id no 8 or its amide, ester or a salts thereof.

Present invention refers to a method for manufacturing of a polypeptide wherein the said method comprises the step of providing the amino acid, synthesizing the amino acid by solid phase or liquid phase synthesis, extraction of polypeptide, purification of the polypeptide

Present invention also provides a method of producing the peptide, precursor or salt thereof which comprises subjecting an amino terminus-constituting amino acid or peptide and a carboxyl terminus-constituting amino acid or peptide to condensation, optionally followed by intramolecular disulfide bond formation.

An embodiment of the invention provides a pharmaceutical composition comprising as the active agent a polypeptide chain or precursor, or pharmaceutically acceptable amide, ester, or a salt thereof wherein said polypeptide chain consists of an amino acid sequence according Seq Id no 1-8.

Present invention also refers to the use of a pharmaceutical composition, comprising as the active agent a polypeptide chain or precursor, or pharmaceutically acceptable amide, ester, or a salt thereof wherein said polypeptide chain consists of an amino acid sequence according Seq Id no 1-8, and can be used as therapeutic polypeptides, targets for drug intervention, ligands to discover relevant targets, biomarkers to monitor diseases.

The invention also provides the use of a peptide, precursor or salt of the invention in the production of an agent for the treatment or prevention of cardio vascular disease, hormone-producing tumors, a hormone secretion inhibitor, a tumor growth inhibitor, which comprises at least one of the amino acid sequence defined under Seq. Id 1 to 8.

An embodiment of the invention also provides an antibody to a polypeptide chain according to paragraph 1 that comprises an amino acid sequences according to Seq Id no 1-8, or its amide, ester or salts thereof. An antibody of the invention can be used also for detecting an inventive polypeptide present in a sample such as a body fluid or a tissue. It may be used also to produce an antibody column for purifying an inventive polypeptide, to detect an inventive polypeptide in each fraction during purification, or to analyze the behavior of an inventive polypeptide in a test cell.

The term “polypeptide” as used herein shall be taken to refer to any polymer consisting of amino acids linked by covalent bonds and this term includes within its scope parts or fragments of full length proteins, such as, for example, peptides, oligopeptides and shorter peptide sequences consisting of at least 2 amino acids, more particularly at least about 5 amino acid residues or more.

The term “polypeptide” includes all moieties containing one or more amino acids linked by a peptide bond. In addition, this term includes within its ambit polymers of modified amino acids, including amino acids which have been post-translationally modified, for example by chemical modification including but not restricted to glycosylation, phosphorylation, acetylation and/or sulphation reactions that effectively alter the basic peptide backbone. Accordingly, a polypeptide may be derived from a naturally-occurring protein, and in particular may be derived from a full-length protein by chemical or enzymatic cleavage, using reagents such as CNBr, or proteases such as trypsin or chymotrypsin, amongst others. Alternatively, such polypeptides may be derived by chemical synthesis using well known peptide synthetic methods. Also included within the scope of the definition of a “polypeptide” are amino acid sequence variants (referred to herein as polypeptide variants). These may contain one or more preferably conservative, amino acid substitutions, deletions, or insertions, in a naturally-occurring amino acid sequence which do not alter at least one essential property of said polypeptide, such as, for example, its biological activity. Such polypeptides may be synthesised by chemical polypeptide synthesis. Conservative amino acid substitutions are well-known in the art. For example, one or more amino acid residues of a native protein can be substituted conservatively with an amino acid residue of similar charge, size or polarity, with the resulting polypeptide retaining functional ability as described herein. Rules for making such substitutions are well known. More specifically, conservative amino acid substitutions are those that generally take place within a family of amino acids that are related in their side chains. Genetically-encoded amino acids are generally divided into four groups: (1) acidic=aspartate, glutamate; (2) basic=lysine, arginine, and histidine; (3) non-polar=alanine, valine, leucine, isoleucine, proline, phenylalanine, methionine, and tryptophan; and (4) unchargedpolar=glycine, asparagine, glutamine, cysteine, serine, threonin, and tyrosine. Phenylalanine, tyrosine and tryptophan are also jointly classified as aromatic amino acids. One or more replacements within any particular group such as, for example, the substitution of leucine for isoleucine or valine are alternatively, the substitution of aspartate for glutamate or threonin for serine, or of any other amino acid residue with a structurally-related amino acid residue will generally have an insignificant effect on the function of the resulting polypeptide.

Included within the scope of the definition of a “polypeptide” are amino acid sequence variants that have undergone unnatural modifications such as but not limited to protection, carboxylation, and derivatization by amide and non-amide bonds as well as covalent and non-covalent modification.

Included in the scope of the definition of the term “polypeptide” is a peptide whose biological activity is predictable as a result of its amino acid sequence corresponding to a functional domain. Also encompassed by the term “polypeptide” is a peptide whose biological activity could not have been predicted by the analysis of its amino acid sequence.

An amino acid is any molecule that contains both amine and carboxylic acid functional groups. Amino acid residue is what is left of an amino acid once a molecule of water has been lost (an H+ from the nitrogenous side and an OH− from the carboxylic side) in the formation of a peptide bond, the chemical bond that links the amino acid monomers in a protein chain. Each protein has its own unique amino acid sequence that is known as its primary structure. Just as the letters of the alphabet can be combined in different ways to form an endless variety of words, amino acids can be linked together in varying sequences to form a huge variety of proteins. The unique shape of each protein determines its function in the body.

A precursor is a substance from which another, usually more active or mature substance is formed. A protein precursor is an inactive protein (or peptide) that can be turned into an active form by posttranslational modification. The name of the precursor for a protein is often prefixed by pro or prepro. Precursors are often used by an organism when the subsequent protein is potentially harmful, but needs to be available on short notice and/or in large quantities.

The Polypeptide, precursors or salts thereof, of the present invention have hormonal activity. Therefore, the polypeptides, precursors and salts of the invention are useful as drugs, for example therapeutic polypeptide, ligand to discover relevant targets (e.g. GPCRs), targets for drug intervention (eg targets for monoclonal antibodies or therelike, receptor fragments), biomarkers to monitor diseases (in combination with tool antibodies to detect peptide fragments in body fluids), protein kinase inhibitors and substrates, T-cell epitopes, Peptide mimotopes of receptor binding sites, Biomarker to measure the expression level.

The DNAs coding for the peptide or precursor of the invention are useful, for example, as agents for the gene therapy or treatment or prevention of cardio vascular disease, hormone-producing tumors, diabetes, gastric ulcer and the like, hormone secretion inhibitors, tumor growth inhibitors, neural activity and so for. Furthermore, the DNAs of the invention are useful as agents for the gene diagnosis of diseases such as cardio vascular disease, hormone-producing tumours, diabetes, gastric ulcer and the like.

A vector is a vehicle for delivering genetic material such as DNA to a cell. DNA by itself may be regarded as a vector, for example in particular when it is used for cell transformation. A vector in this sense is a DNA construct, such as a plasmid or a bacterial artificial chromosome that contains an origin of replication. An appropriate replication origin causes a cell to copy the construct along with the cell's chromosomes and to pass it along to its progeny. A single cell that has been transformed with a vector will grow into an entire culture of cells, which all contain the vector, as well as any gene attached to it within the construct. Because the constructs can be extracted from the cells by purification techniques, transformation with a vector is a way of making a small number of DNA molecules in to a much larger one. A vector can be a E. coli-derived plasmid (e.g., pBR322, pBR325, pUC12, pUC13), a Bacillus subtilis-derived plasmid (e.g., pUB110, pTP5, pC194), a yeast-derived plasmid (e.g., pSH19, pSH15), a bacteriophage such as [lambda] phage, as well as an animal virus such as retrovirus, vaccinia virus, vaculovirus and the like.

The antibodies against the peptide, precursor or salt of the invention can specifically recognize the peptide, precursor or salt of the invention. It may be used also to produce an antibody column for purifying an inventive polypeptide, to detect an inventive polypeptide in each fraction during purification, or to analyze the behavior of a novel polypeptide in a test cell. Hence it can be used for assaying the peptide or equivalent of the invention in test solutions.

### Results

### 1.0 Description of the Computer Programs:

### 1.1 Signal P Version 2.0

Objective: This program was used to detect potential signal sequences, it was used with a cut off score of 0.98. Signal P version 2.0 predicts the presence and location of signal peptide cleavage sites in amino acid sequences from different organisms: The method incorporates a prediction of cleavage sites and a signal peptide/non-signal peptide prediction based on a combination of several artificial neural networks and hidden Markov models.

### 1.2 TMHMM Version 2.0

Objective: This program was used to define possible membrane spanning regions in protein sequences. TMHMM version 2.0 is used for prediction of transmembrane helices in proteins. Predicted TM segments in the n-terminal region sometime turn out to be signal peptides.

### 1.3 ProP Version 1.0

Objective: This program was used to detect potential cleavage sites in protein sequences. It was used with a score of 0.09. This program predicts arginine and lysine propeptide cleavage sites in eukaryotic protein sequences using an ensemble of neural networks. Furin-specific prediction is the default. It is also possible to perform a general proprotein convertase (PC) prediction. This program is integrated with the SignalP program predicting the presence and location of signal peptide cleavage sites.

1.4 InterPro Version 12 in Combination with InterProScan

InterPro is a database of protein families, domains and functional sites in which identifiable features found in known proteins can be applied to unknown protein sequences. 1.5 InterProScan is the program used to compare amino acid sequences to the InterPro database.

### 1.6 BLAST Version 2.2.9

The Basic Local Alignment Search Tool (BLAST) finds regions of local similarity between sequences. The program compares nucleotide or protein sequences to sequence databases and calculates the statistical significance of matches. BLAST can be used to infer functional and evolutionary relationships between sequences as well as help identify members of gene families. BLAST uses Karlin-Altschul Statistics to determine the statistical significance of the alignments it produces. The basic algorithm can be implemented in a number of ways and applied in a variety of contexts including straightforward DNA and protein sequence database searches, motif searches, gene identification searches, and in the analysis of multiple regions of similarity in long DNA sequences. In addition to its flexibility and tractability to mathematical analysis, BLAST is an order of magnitude faster than existing sequence comparison tools of comparable sensitivity.

All these programs as mentioned before are accessible via the public domain internet

The biological role of the hormone peptides of the invention have been examined by means of expression profile studies (FIGS. 1 to 6). The presence of hormone peptides could be demonstrated for several tissues to be different depending on the particular sequence. The differential expression of hormone peptides according to the invention in different tissues is regarded as strong indication for their important biological role. The adjustment of the concentration of one or several of the hormone peptides of the invention by increasing or diminishing the amount of such hormone peptides could have a strong impact to the treatment of a disease in need of diminished or elevated concentrations of one or several of such hormone peptides (e.g. cardiovascular disease, metabolic disease, mental illness, cancer, viral, bacterial or yeast triggered infections and others).

### 2.0 Expression Profiles

### 2.1) Performing the Tissue Expression Analysis

The tissue expression analysis was performed by means of TaqMan gene expression analyses

PCR (Polymerase Chain Reaction) is a standard technique in medical and biological research labs for a variety of tasks, such as the detection of hereditary diseases, the identification of genetic fingerprints, the diagnosis of infectious diseases, the cloning of genes, paternity testing, and DNA computing.

Quantitative PCR is used to rapidly measure the quantity of PCR product (preferably real-time), thus is an indirect method for quantitatively measuring starting amounts of DNA, cDNA or RNA. This is commonly used for the purpose of determining whether a sequence is present or not, and if it is present the number of copies in the sample. Description of protocol used for gene expression studies in present invention:

### Sample Preparation:

RNA samples from the tissues tested were purchased from different vendors.

### DNase Digest:

All products were purchased from Ambion. DNA free DNase treatment and removal reagents (1906). DNase digest was performed using 50 μg RNA and according to the manufacturer's protocol.

cDNA Synthesis:

Products for cDNA were purchased from Applera Reverse Transcriptase Kit (N8080234), RNase Inhibitor (N8080119). cDNA Synthesis from 10 μg RNA was performed.

### TaqMan Reaction Setup:

Products for the PCR were purchased from Applera. TaqMan Universal PCR Master Mix (4305719) was used in each reaction. Target forward Primer, Target reverse Primer, Target probe labeled with FAM for each gene, respectively (see Table with primer sequences). PCR was performed using the ABI Prism 7900 (Applera) under the following PCR conditions: 2 minutes at 50° C., 10 minutes at 95° C., 40 cycles with 95° C. for 15 s and 1 minute at 60° C. PCR was set up as a Multiplex PCR using B2M as endogenous control for normalisation.

2.3) The following expression profiles could be gained for a gene encoding a peptide sequence according to Seq ID Nr 1 (FIG. 7). These results are exhibited as well in FIG. 1 however in another format:

| Tissue Type | Mean | RPL37aMean | ∂∂ Ct | Expression |
|---|---|---|---|---|
| Fetal Brain | 30.96 | 24.22 | 6.74 | 9.34 |
| Brain | 30.01 | 22.31 | 7.70 | 4.80 |
| Brain Hippocampus | 31.58 | 21.59 | 9.99 | 0.98 |
| Cerebellum | 28.74 | 22.28 | 6.47 | 11.29 |
| Brain Cortex Temporal | 30.15 | 23.05 | 7.10 | 7.29 |
| Brain Cortex Frontal | 32.02 | 22.81 | 9.20 | 1.70 |
| Substantia Nigra | 31.09 | 21.01 | 10.08 | 0.93 |
| Dorsal Root Ganglion | 33.87 | 21.16 | 12.72 | 0.15 |
| Spinal Cord | 34.57 | 21.28 | 13.30 | 0.10 |
| Hypothalamus | 32.69 | 21.39 | 11.29 | 0.40 |
| Amygdala | 31.25 | 21.07 | 10.18 | 0.86 |
| Adrenal Gland | 40.00 | 20.68 | 19.32 | 0.00 |
| Salivary Gland | 32.97 | 21.03 | 11.94 | 0.25 |
| Thyroid | 31.73 | 20.32 | 11.41 | 0.37 |
| Heart | 34.09 | 19.87 | 14.22 | 0.05 |
| Aorta | 34.96 | 20.66 | 14.30 | 0.05 |
| Fetal Aorta | 28.18 | 22.46 | 5.72 | 18.95 |
| AoSMC | 34.27 | 20.34 | 13.93 | 0.06 |
| HUVEC | 40.00 | 20.89 | 19.11 | 0.00 |
| Adipose | 33.16 | 19.58 | 13.58 | 0.08 |
| Fetal Liver | 34.26 | 21.87 | 12.39 | 0.19 |
| Liver | 34.00 | 20.38 | 13.62 | 0.08 |
| Normal Spleen | 34.15 | 18.70 | 15.45 | 0.02 |
| Tonsil | 33.53 | 18.82 | 14.71 | 0.04 |
| Bone Marrow | 36.58 | 21.18 | 15.40 | 0.00 |
| Thymus | 30.26 | 19.67 | 10.58 | 0.65 |
| PBMC Control | 39.54 | 17.77 | 21.77 | 0.00 |
| PBMC, PHA treated | 35.60 | 17.25 | 18.35 | 0.00 |
| THP-1 monocytes | 28.39 | 20.18 | 8.21 | 3.38 |
| THP-1 macrophages | 31.77 | 19.39 | 12.38 | 0.19 |
| Kidney | 34.59 | 21.75 | 12.84 | 0.14 |
| Skeletal Muscle | 24.83 | 21.92 | 2.91 | 133.02 |
| Skin | 30.39 | 20.97 | 9.43 | 1.45 |
| Chondrocytes | 30.97 | 20.51 | 10.46 | 0.71 |
| Trachea | 34.31 | 20.35 | 13.96 | 0.06 |
| Fetal Lung | 31.75 | 22.06 | 9.69 | 1.21 |
| Lung | 30.90 | 19.50 | 11.40 | 0.37 |
| Lung COPD Control | 40.00 | 20.36 | 19.64 | 0.00 |
| Lung COPD | 35.81 | 20.15 | 15.66 | 0.00 |
| Lung Tumor Control | 35.20 | 18.65 | 16.55 | 0.00 |
| Lung Tumor | 34.80 | 18.86 | 15.93 | 0.02 |
| (adenocarcinoma) | | | | |
| Lung Tumor (carcinoma) | 32.71 | 19.29 | 13.42 | 0.09 |
| A549 cells | 25.55 | 19.50 | 6.05 | 15.06 |
| Breast | 32.86 | 21.84 | 11.02 | 0.48 |
| Breast Tumor Control | 34.14 | 20.87 | 13.27 | 0.10 |
| Breast | 33.85 | 20.24 | 13.60 | 0.08 |
| Tumor (adenocarcinoma) | | | | |
| Mammary Gland | 37.21 | 20.65 | 16.56 | 0.00 |
| Colon | 35.71 | 19.86 | 15.85 | 0.00 |
| Colon IBD Control | 37.82 | 19.17 | 18.65 | 0.00 |
| Colon IBD | 38.87 | 19.39 | 19.48 | 0.00 |
| Colon Tumor Control | 37.41 | 19.70 | 17.71 | 0.00 |
| Colon | 36.40 | 18.50 | 17.90 | 0.00 |
| Tumor (adenocarcinoma) | | | | |
| Esophagus | 28.07 | 22.34 | 5.73 | 18.88 |
| Stomach | 38.35 | 20.98 | 17.37 | 0.00 |
| Small Intestine | 33.91 | 21.20 | 12.71 | 0.15 |
| Pancreas | 40.00 | 23.63 | 16.37 | 0.00 |
| Ovary | 37.02 | 21.12 | 15.89 | 0.00 |
| Ovary Tumor Control | 35.67 | 20.46 | 15.22 | 0.00 |
| Ovary | 31.79 | 19.67 | 12.12 | 0.22 |
| Tumor (adenocarcinoma) | | | | |
| Ovary Tumor (carcinoma) | 31.47 | 18.92 | 12.55 | 0.17 |
| Prostate | 29.26 | 20.15 | 9.11 | 1.81 |
| Prostate Tumor Control | 40.00 | 20.60 | 19.40 | 0.00 |
| Prostate | 35.15 | 20.15 | 15.00 | 0.00 |
| Tumor (adenocarcinoma) | | | | |
| HeLa cells | 34.97 | 19.45 | 15.51 | 0.02 |
| Uterus | 36.13 | 20.32 | 15.81 | 0.00 |
| Placenta | 32.62 | 22.18 | 10.44 | 0.72 |
| Testis | 29.85 | 22.58 | 7.27 | 6.46 |
| Positive Control (HIPCO) | 29.32 | 20.27 | 9.05 | 1.88 |

2.4) The following expression profiles could be gained for a gene encoding a peptide sequence according to Seq ID Nr 2 (FIG. 8). These results are exhibited as well in FIG. 2 however in another format:

| Tissue Type | Mean | RPL37aMean | ∂∂ Ct | Expression |
|---|---|---|---|---|
| Fetal Brain | 39.66 | 24.66 | 14.99 | 0.00 |
| Brain | 34.18 | 22.90 | 11.29 | 0.40 |
| Brain Hippocampus | 33.12 | 21.91 | 11.21 | 0.42 |
| Cerebellum | 40.00 | 22.84 | 17.16 | 0.00 |
| Brain Cortex Temporal | 33.28 | 23.61 | 9.66 | 1.23 |
| Brain Cortex Frontal | 34.81 | 23.20 | 11.61 | 0.32 |
| Substantia Nigra | 40.00 | 21.39 | 18.61 | 0.00 |
| Dorsal Root Ganglion | 39.83 | 21.35 | 18.47 | 0.00 |
| Spinal Cord | 39.55 | 21.57 | 17.97 | 0.00 |
| Hypothalamus | 35.65 | 21.83 | 13.82 | 0.00 |
| Amygdala | 35.82 | 21.52 | 14.30 | 0.00 |
| Adrenal Gland | 40.00 | 21.09 | 18.91 | 0.00 |
| Salivary Gland | 40.00 | 21.56 | 18.44 | 0.00 |
| Thyroid | 40.00 | 20.52 | 19.48 | 0.00 |
| Heart | 34.96 | 20.69 | 14.26 | 0.05 |
| Aorta | 35.55 | 20.89 | 14.65 | 0.00 |
| Fetal Aorta | 35.80 | 22.86 | 12.94 | 0.00 |
| AoSMC | 29.23 | 19.25 | 9.98 | 0.99 |
| HUVEC | 40.00 | 21.28 | 18.72 | 0.00 |
| Adipose | 39.40 | 19.77 | 19.63 | 0.00 |
| Fetal Liver | 39.98 | 22.09 | 17.89 | 0.00 |
| Liver | 40.00 | 20.66 | 19.34 | 0.00 |
| Normal Spleen | 32.16 | 19.05 | 13.11 | 0.11 |
| Tonsil | 35.12 | 19.12 | 15.99 | 0.00 |
| Bone Marrow | 39.75 | 21.57 | 18.18 | 0.00 |
| Thymus | 38.16 | 20.01 | 18.15 | 0.00 |
| PBMC Control | 39.86 | 18.29 | 21.57 | 0.00 |
| PBMC, PHA treated | 39.83 | 18.11 | 21.72 | 0.00 |
| THP-1 monocytes | 39.62 | 20.59 | 19.03 | 0.00 |
| THP-1 macrophages | 39.80 | 19.82 | 19.98 | 0.00 |
| Kidney | 40.00 | 22.12 | 17.88 | 0.00 |
| Skeletal Muscle | 39.30 | 22.27 | 17.02 | 0.00 |
| Skin | 39.49 | 21.09 | 18.40 | 0.00 |
| Chondrocytes | 34.97 | 20.53 | 14.44 | 0.04 |
| Trachea | 39.64 | 20.70 | 18.94 | 0.00 |
| Fetal Lung | 37.09 | 22.44 | 14.65 | 0.00 |
| Lung | 34.54 | 19.60 | 14.93 | 0.03 |
| Lung COPD Control | 40.00 | 20.04 | 19.96 | 0.00 |
| Lung COPD | 39.00 | 20.28 | 18.72 | 0.00 |
| Lung Tumor Control | 35.62 | 18.79 | 16.83 | 0.00 |
| Lung | 35.29 | 18.81 | 16.47 | 0.00 |
| Tumor (adenocarcinoma) | | | | |
| Lung Tumor (carcinoma) | 34.02 | 19.18 | 14.85 | 0.03 |
| A549 cells | 39.81 | 19.75 | 20.06 | 0.00 |
| Breast | 35.69 | 21.97 | 13.72 | 0.00 |
| Breast Tumor Control | 31.53 | 20.88 | 10.65 | 0.62 |
| Breast | 31.49 | 20.02 | 11.47 | 0.35 |
| Tumor (adenocarcinoma) | | | | |
| Mammary Gland | 32.62 | 20.40 | 12.21 | 0.21 |
| Colon | 38.69 | 20.05 | 18.64 | 0.00 |
| Colon IBD Control | 35.60 | 19.02 | 16.58 | 0.00 |
| Colon IBD | 35.39 | 19.50 | 15.89 | 0.00 |
| Colon Tumor Control | 35.60 | 19.63 | 15.97 | 0.00 |
| Colon | 35.25 | 19.32 | 15.93 | 0.00 |
| Tumor (adenocarcinoma) | | | | |
| Esophagus | 37.59 | 21.90 | 15.68 | 0.00 |
| Stomach | 35.13 | 20.88 | 14.25 | 0.00 |
| Small Intestine | 39.54 | 21.00 | 18.54 | 0.00 |
| Pancreas | 40.00 | 23.39 | 16.61 | 0.00 |
| Ovary | 39.94 | 21.12 | 18.81 | 0.00 |
| Ovary Tumor Control | 39.50 | 20.65 | 18.85 | 0.00 |
| Ovary | 36.06 | 19.50 | 16.56 | 0.00 |
| Tumor (adenocarcinoma) | | | | |
| Ovary Tumor (carcinoma) | 39.67 | 18.95 | 20.72 | 0.00 |
| Prostate | 39.59 | 20.37 | 19.22 | 0.00 |
| Prostate Tumor Control | 40.00 | 20.54 | 19.46 | 0.00 |
| Prostate | 37.51 | 20.27 | 17.23 | 0.00 |
| Tumor (adenocarcinoma) | | | | |
| HeLa cells | 40.00 | 19.34 | 20.66 | 0.00 |
| Uterus | 32.39 | 20.46 | 11.93 | 0.26 |
| Placenta | 35.74 | 22.01 | 13.73 | 0.00 |
| Testis | 32.42 | 22.53 | 9.89 | 1.06 |
| Positive Control (HIPCO) | 33.15 | 20.48 | 12.67 | 0.15 |

2.5) The following expression profiles could be gained for a gene encoding a peptide sequence according to Seq ID Nr 3 (FIG. 9). These results are exhibited as well in FIG. 3 however in another format:

| Tissue Type | Mean | RPL37aMean | ∂∂ Ct | Expression |
|---|---|---|---|---|
| Fetal Brain | 27.61 | 23.88 | 3.73 | 75.43 |
| Brain | 27.58 | 21.82 | 5.75 | 18.56 |
| Brain Hippocampus | 26.95 | 21.09 | 5.86 | 17.23 |
| Cerebellum | 26.98 | 21.83 | 5.15 | 28.14 |
| Brain Cortex Temporal | 27.87 | 22.74 | 5.13 | 28.48 |
| Brain Cortex Frontal | 27.92 | 22.36 | 5.57 | 21.10 |
| Substantia Nigra | 26.03 | 20.48 | 5.55 | 21.34 |
| Dorsal Root Ganglion | 24.93 | 20.79 | 4.14 | 56.74 |
| Spinal Cord | 25.90 | 20.67 | 5.22 | 26.80 |
| Hypothalamus | 26.44 | 20.97 | 5.46 | 22.68 |
| Amygdala | 26.70 | 20.75 | 5.95 | 16.20 |
| Adrenal Gland | 26.30 | 20.37 | 5.93 | 16.43 |
| Salivary Gland | 27.34 | 20.91 | 6.42 | 11.67 |
| Thyroid | 25.47 | 19.95 | 5.52 | 21.79 |
| Heart | 24.93 | 20.06 | 4.87 | 34.20 |
| Aorta | 25.44 | 20.08 | 5.36 | 24.31 |
| Fetal Aorta | 24.71 | 22.16 | 2.56 | 169.83 |
| AoSMC | 25.32 | 20.02 | 5.30 | 25.40 |
| HUVEC | 25.24 | 20.32 | 4.91 | 33.18 |
| Adipose | 23.86 | 19.20 | 4.66 | 39.64 |
| Fetal Liver | 26.93 | 21.41 | 5.52 | 21.82 |
| Liver | 27.72 | 20.07 | 7.65 | 4.98 |
| Normal Spleen | 25.66 | 18.39 | 7.27 | 6.47 |
| Tonsil | 26.10 | 18.32 | 7.78 | 4.54 |
| Bone Marrow | 29.36 | 20.50 | 8.86 | 2.15 |
| Thymus | 25.00 | 18.95 | 6.04 | 15.16 |
| PBMC Control | 28.79 | 17.48 | 11.32 | 0.39 |
| PBMC, PHA treated | 24.68 | 17.27 | 7.41 | 5.88 |
| THP-1 monocytes | 30.34 | 19.53 | 10.82 | 0.55 |
| THP-1 macrophages | 30.35 | 18.95 | 11.41 | 0.37 |
| Kidney | 27.82 | 21.35 | 6.47 | 11.25 |
| Skeletal Muscle | 26.66 | 21.31 | 5.35 | 24.50 |
| Skin | 26.02 | 20.39 | 5.63 | 20.19 |
| Chondrocytes | 30.08 | 19.75 | 10.33 | 0.78 |
| Trachea | 25.64 | 20.58 | 5.06 | 29.98 |
| Fetal Lung | 23.50 | 22.27 | 1.22 | 428.06 |
| Lung | 24.92 | 19.39 | 5.53 | 21.58 |
| Lung COPD Control | 26.36 | 19.70 | 6.66 | 9.87 |
| Lung COPD | 25.89 | 20.11 | 5.78 | 18.18 |
| Lung Tumor Control | 24.46 | 18.90 | 5.56 | 21.24 |
| Lung | 25.60 | 18.75 | 6.85 | 8.67 |
| Tumor (adenocarcinoma) | | | | |
| Lung Tumor (carcinoma) | 25.13 | 19.40 | 5.72 | 18.93 |
| A549 cells | 29.30 | 19.48 | 9.82 | 1.11 |
| Breast | 27.63 | 21.93 | 5.70 | 19.27 |
| Breast Tumor Control | 24.15 | 20.82 | 3.33 | 99.60 |
| Breast | 25.02 | 19.88 | 5.13 | 28.52 |
| Tumor (adenocarcinoma) | | | | |
| Mammary Gland | 23.96 | 20.32 | 3.64 | 80.12 |
| Colon | 25.25 | 19.76 | 5.49 | 22.28 |
| Colon IBD Control | 25.44 | 18.98 | 6.46 | 11.35 |
| Colon IBD | 26.17 | 19.58 | 6.59 | 10.35 |
| Colon Tumor Control | 24.99 | 19.50 | 5.50 | 22.15 |
| Colon | 25.50 | 19.15 | 6.35 | 12.25 |
| Tumor (adenocarcinoma) | | | | |
| Esophagus | 26.87 | 21.94 | 4.93 | 32.88 |
| Stomach | 25.74 | 21.13 | 4.61 | 40.88 |
| Small Intestine | 25.46 | 21.08 | 4.38 | 47.96 |
| Pancreas | 29.00 | 23.14 | 5.85 | 17.28 |
| Ovary | 24.37 | 20.93 | 3.44 | 92.12 |
| Ovary Tumor Control | 22.48 | 20.62 | 1.86 | 275.88 |
| Ovary | 26.19 | 19.48 | 6.71 | 9.54 |
| Tumor (adenocarcinoma) | | | | |
| Ovary Tumor (carcinoma) | 26.29 | 19.21 | 7.08 | 7.37 |
| Prostate | 25.44 | 20.35 | 5.08 | 29.46 |
| Prostate Tumor Control | 25.41 | 20.59 | 4.82 | 35.38 |
| Prostate | 25.14 | 20.09 | 5.05 | 30.28 |
| Tumor (adenocarcinoma) | | | | |
| HeLa cells | 29.11 | 19.35 | 9.75 | 1.16 |
| Uterus | 23.77 | 20.27 | 3.50 | 88.49 |
| Placenta | 27.73 | 22.13 | 5.59 | 20.74 |
| Testis | 25.44 | 22.28 | 3.16 | 111.93 |
| Positive Control (HIPCO) | 26.57 | 20.50 | 6.07 | 14.88 |

2.6) The following expression profiles could be gained for a gene encoding a peptide sequence according to Seq ID Nr 4 (FIG. 10). These results are exhibited as well in FIG. 4 however in another format:

| Tissue Type | Mean | RPL37aMean | ∂∂ Ct | Expression |
|---|---|---|---|---|
| Fetal Brain | 26.65 | 24.42 | 2.23 | 213.09 |
| Brain | 25.81 | 22.65 | 3.16 | 111.58 |
| Brain Hippocampus | 25.46 | 21.68 | 3.78 | 72.68 |
| Cerebellum | 24.21 | 22.63 | 1.58 | 333.75 |
| Brain Cortex Temporal | 26.22 | 23.25 | 2.97 | 127.57 |
| Brain Cortex Frontal | 25.85 | 23.02 | 2.84 | 139.77 |
| Substantia Nigra | 25.09 | 21.41 | 3.68 | 77.94 |
| Dorsal Root Ganglion | 26.31 | 21.40 | 4.91 | 33.21 |
| Spinal Cord | 25.54 | 21.21 | 4.32 | 49.99 |
| Hypothalamus | 25.34 | 21.69 | 3.65 | 79.80 |
| Amygdala | 25.59 | 21.41 | 4.18 | 55.15 |
| Adrenal Gland | 26.43 | 20.92 | 5.51 | 21.96 |
| Salivary Gland | 27.09 | 21.26 | 5.83 | 17.58 |
| Thyroid | 25.10 | 20.54 | 4.56 | 42.30 |
| Heart | 25.53 | 20.64 | 4.89 | 33.68 |
| Aorta | 23.48 | 20.64 | 2.84 | 140.12 |
| Fetal Aorta | 25.76 | 22.81 | 2.95 | 129.46 |
| AoSMC | 24.74 | 20.22 | 4.51 | 43.84 |
| HUVEC | 27.69 | 21.06 | 6.63 | 10.08 |
| Adipose | 24.15 | 19.53 | 4.61 | 40.82 |
| Fetal Liver | 26.81 | 21.91 | 4.90 | 33.43 |
| Liver | 27.39 | 20.57 | 6.82 | 8.85 |
| Normal Spleen | 26.36 | 18.93 | 7.43 | 5.82 |
| Tonsil | 25.40 | 19.02 | 6.38 | 12.02 |
| Bone Marrow | 27.72 | 21.20 | 6.52 | 10.89 |
| Thymus | 26.13 | 19.67 | 6.46 | 11.35 |
| PBMC Control | 24.51 | 18.28 | 6.23 | 13.33 |
| PBMC, PHA treated | 26.50 | 17.83 | 8.67 | 2.46 |
| THP-1 monocytes | 27.34 | 20.44 | 6.90 | 8.38 |
| THP-1 macrophages | 25.31 | 19.42 | 5.89 | 16.91 |
| Kidney | 26.42 | 21.98 | 4.44 | 46.00 |
| Skeletal Muscle | 26.83 | 21.82 | 5.01 | 31.00 |
| Skin | 26.37 | 20.87 | 5.50 | 22.07 |
| Chondrocytes | 25.32 | 20.37 | 4.95 | 32.35 |
| Trachea | 26.88 | 20.68 | 6.20 | 13.63 |
| Fetal Lung | 24.27 | 22.12 | 2.15 | 224.85 |
| Lung | 24.34 | 19.66 | 4.68 | 38.99 |
| Lung COPD Control | 25.91 | 20.04 | 5.87 | 17.08 |
| Lung COPD | 25.86 | 20.43 | 5.43 | 23.27 |
| Lung Tumor Control | 24.26 | 18.90 | 5.37 | 24.23 |
| Lung | 24.89 | 18.76 | 6.13 | 14.26 |
| Tumor (adenocarcinoma) | | | | |
| Lung Tumor (carcinoma) | 24.62 | 19.34 | 5.29 | 25.61 |
| A549 cells | 24.30 | 19.69 | 4.61 | 40.86 |
| Breast | 26.25 | 21.79 | 4.47 | 45.26 |
| Breast Tumor Control | 25.39 | 21.00 | 4.39 | 47.72 |
| Breast | 25.06 | 19.91 | 5.15 | 28.22 |
| Tumor (adenocarcinoma) | | | | |
| Mammary Gland | 25.45 | 20.47 | 4.99 | 31.54 |
| Colon | 25.67 | 19.89 | 5.78 | 18.14 |
| Colon IBD Control | 24.98 | 19.18 | 5.80 | 17.95 |
| Colon IBD | 26.45 | 19.46 | 6.98 | 7.91 |
| Colon Tumor Control | 24.93 | 19.69 | 5.25 | 26.32 |
| Colon | 25.21 | 19.41 | 5.80 | 17.93 |
| Tumor (adenocarcinoma) | | | | |
| Esophagus | 27.85 | 22.07 | 5.78 | 18.21 |
| Stomach | 26.33 | 20.90 | 5.43 | 23.22 |
| Small Intestine | 26.52 | 21.21 | 5.31 | 25.24 |
| Pancreas | 28.54 | 23.50 | 5.03 | 30.53 |
| Ovary | 26.27 | 21.12 | 5.15 | 28.07 |
| Ovary Tumor Control | 24.96 | 20.85 | 4.11 | 57.84 |
| Ovary | 25.29 | 19.54 | 5.76 | 18.51 |
| Tumor (adenocarcinoma) | | | | |
| Ovary Tumor (carcinoma) | 25.58 | 19.23 | 6.35 | 12.28 |
| Prostate | 25.83 | 20.29 | 5.55 | 21.42 |
| Prostate Tumor Control | 25.97 | 20.73 | 5.25 | 26.31 |
| Prostate | 25.30 | 20.36 | 4.94 | 32.52 |
| Tumor (adenocarcinoma) | | | | |
| HeLa cells | 24.16 | 19.70 | 4.46 | 45.58 |
| Uterus | 25.44 | 20.46 | 4.98 | 31.72 |
| Placenta | 24.65 | 22.12 | 2.53 | 172.96 |
| Testis | 27.35 | 22.27 | 5.08 | 29.60 |
| Positive Control (HIPCO) | 24.97 | 20.42 | 4.55 | 42.65 |

2.7) The following expression profiles could be gained for a gene encoding a peptide sequence according to Seq ID Nr 5 (FIG. 11). These results are exhibited as well in FIG. 5 however in another format:

| Tissue Type | Mean | RPL37aMean | ∂∂ Ct | Expression |
|---|---|---|---|---|
| Fetal Brain | 38.36 | 23.87 | 14.49 | 0.00 |
| Brain | 30.39 | 22.28 | 8.12 | 3.60 |
| Brain Hippocampus | 32.35 | 21.47 | 10.88 | 0.53 |
| Cerebellum | 33.44 | 22.32 | 11.12 | 0.45 |
| Brain Cortex Temporal | 30.48 | 22.87 | 7.61 | 5.12 |
| Brain Cortex Frontal | 29.54 | 22.87 | 6.67 | 9.85 |
| Substantia Nigra | 29.00 | 20.87 | 8.13 | 3.57 |
| Dorsal Root Ganglion | 33.57 | 21.09 | 12.48 | 0.18 |
| Spinal Cord | 34.83 | 21.18 | 13.65 | 0.08 |
| Hypothalamus | 33.30 | 21.15 | 12.14 | 0.22 |
| Amygdala | 29.81 | 21.10 | 8.71 | 2.39 |
| Adrenal Gland | 35.88 | 20.63 | 15.25 | 0.00 |
| Salivary Gland | 32.11 | 20.86 | 11.25 | 0.41 |
| Thyroid | 30.02 | 20.01 | 10.01 | 0.97 |
| Heart | 22.92 | 20.24 | 2.68 | 155.99 |
| Aorta | 32.01 | 20.35 | 11.66 | 0.31 |
| Fetal Aorta | 32.25 | 22.20 | 10.05 | 0.95 |
| AoSMC | 36.69 | 20.10 | 16.59 | 0.00 |
| HUVEC | 37.96 | 20.81 | 17.15 | 0.00 |
| Adipose | 31.88 | 19.24 | 12.64 | 0.16 |
| Fetal Liver | 35.04 | 21.66 | 13.38 | 0.00 |
| Liver | 35.58 | 20.23 | 15.35 | 0.00 |
| Normal Spleen | 39.90 | 18.59 | 21.31 | 0.00 |
| Tonsil | 36.19 | 18.63 | 17.56 | 0.00 |
| Bone Marrow | 37.40 | 21.07 | 16.33 | 0.00 |
| Thymus | 39.63 | 19.29 | 20.34 | 0.00 |
| PBMC Control | 39.28 | 17.90 | 21.39 | 0.00 |
| PBMC, PHA treated | 39.82 | 17.58 | 22.23 | 0.00 |
| THP-1 monocytes | 39.31 | 19.99 | 19.32 | 0.00 |
| THP-1 macrophages | 37.87 | 19.08 | 18.80 | 0.00 |
| Kidney | 34.05 | 21.20 | 12.85 | 0.14 |
| Skeletal Muscle | 23.72 | 21.72 | 2.00 | 249.66 |
| Skin | 32.47 | 20.76 | 11.70 | 0.30 |
| Chondrocytes | 32.69 | 20.10 | 12.59 | 0.16 |
| Trachea | 34.91 | 20.45 | 14.46 | 0.04 |
| Fetal Lung | 38.69 | 22.27 | 16.42 | 0.00 |
| Lung | 33.40 | 19.59 | 13.81 | 0.07 |
| Lung COPD Control | 38.12 | 19.92 | 18.20 | 0.00 |
| Lung COPD | 39.60 | 20.44 | 19.16 | 0.00 |
| Lung Tumor Control | 35.64 | 19.07 | 16.57 | 0.00 |
| Lung | 36.67 | 18.41 | 18.26 | 0.00 |
| Tumor (adenocarcinoma) | | | | |
| Lung Tumor (carcinoma) | 36.89 | 19.51 | 17.38 | 0.00 |
| A549 cells | 37.89 | 19.86 | 18.03 | 0.00 |
| Breast | 30.08 | 21.84 | 8.24 | 3.32 |
| Breast Tumor Control | 38.42 | 21.05 | 17.36 | 0.00 |
| Breast | 40.00 | 20.29 | 19.71 | 0.00 |
| Tumor (adenocarcinoma) | | | | |
| Mammary Gland | 37.00 | 20.21 | 16.80 | 0.00 |
| Colon | 38.67 | 19.93 | 18.74 | 0.00 |
| Colon IBD Control | 39.98 | 19.15 | 20.84 | 0.00 |
| Colon IBD | 38.63 | 19.84 | 18.79 | 0.00 |
| Colon Tumor Control | 39.68 | 19.82 | 19.87 | 0.00 |
| Colon | 39.60 | 19.26 | 20.34 | 0.00 |
| Tumor (adenocarcinoma) | | | | |
| Esophagus | 28.60 | 22.24 | 6.36 | 12.16 |
| Stomach | 36.43 | 21.12 | 15.31 | 0.00 |
| Small Intestine | 38.13 | 21.11 | 17.02 | 0.00 |
| Pancreas | 40.00 | 23.71 | 16.29 | 0.00 |
| Ovary | 34.51 | 21.07 | 13.43 | 0.09 |
| Ovary Tumor Control | 32.33 | 20.61 | 11.72 | 0.30 |
| Ovary | 39.25 | 19.62 | 19.63 | 0.00 |
| Tumor (adenocarcinoma) | | | | |
| Ovary Tumor (carcinoma) | 37.45 | 19.11 | 18.34 | 0.00 |
| Prostate | 32.12 | 20.34 | 11.79 | 0.28 |
| Prostate Tumor Control | 39.62 | 20.71 | 18.92 | 0.00 |
| Prostate | 38.21 | 20.38 | 17.83 | 0.00 |
| Tumor (adenocarcinoma) | | | | |
| HeLa cells | 40.00 | 19.38 | 20.62 | 0.00 |
| Uterus | 33.22 | 20.19 | 13.03 | 0.12 |
| Placenta | 39.92 | 22.32 | 17.59 | 0.00 |
| Testis | 33.43 | 22.25 | 11.18 | 0.43 |
| Positive Control (HIPCO) | 33.10 | 20.69 | 12.42 | 0.18 |

2.8) The following expression profiles could be gained for a gene encoding a peptide sequence according to Seq ID Nr 6 (FIG. 12). These results are exhibited as well in FIG. 6 however in another format:

| Tissue Type | Mean | RPL37aMean | ∂∂ Ct | Expression |
|---|---|---|---|---|
| Fetal Brain | 27.34 | 25.41 | 1.93 | 263.22 |
| Brain | 29.19 | 23.30 | 5.88 | 16.95 |
| Brain Hippocampus | 28.80 | 22.54 | 6.26 | 13.07 |
| Cerebellum | 32.16 | 23.37 | 8.79 | 2.26 |
| Brain Cortex Temporal | 29.79 | 24.09 | 5.71 | 19.12 |
| Brain Cortex Frontal | 29.28 | 24.02 | 5.27 | 25.99 |
| Substantia Nigra | 28.03 | 22.16 | 5.87 | 17.05 |
| Dorsal Root Ganglion | 29.51 | 22.02 | 7.50 | 5.53 |
| Spinal Cord | 29.63 | 22.18 | 7.45 | 5.72 |
| Hypothalamus | 28.81 | 22.42 | 6.38 | 11.99 |
| Amygdala | 28.93 | 22.13 | 6.80 | 9.00 |
| Adrenal Gland | 33.43 | 21.62 | 11.80 | 0.28 |
| Salivary Gland | 34.15 | 22.61 | 11.53 | 0.34 |
| Thyroid | 32.85 | 21.28 | 11.57 | 0.33 |
| Heart | 29.36 | 21.31 | 8.05 | 3.76 |
| Aorta | 31.93 | 21.35 | 10.58 | 0.65 |
| Fetal Aorta | 28.86 | 23.35 | 5.51 | 21.88 |
| AoSMC | 27.84 | 21.18 | 6.66 | 9.92 |
| HUVEC | 30.12 | 21.89 | 8.23 | 3.33 |
| Adipose | 30.83 | 20.28 | 10.54 | 0.67 |
| Fetal Liver | 29.63 | 22.65 | 6.98 | 7.91 |
| Liver | 28.50 | 21.28 | 7.22 | 6.70 |
| Normal Spleen | 31.40 | 19.53 | 11.86 | 0.27 |
| Tonsil | 32.57 | 19.71 | 12.86 | 0.13 |
| Bone Marrow | 35.75 | 22.25 | 13.50 | 0.00 |
| Thymus | 30.85 | 20.87 | 9.98 | 0.99 |
| PBMC Control | 34.92 | 18.96 | 15.96 | 0.02 |
| PBMC, PHA treated | 37.76 | 18.82 | 18.93 | 0.00 |
| THP-1 monocytes | 32.76 | 21.11 | 11.65 | 0.31 |
| THP-1 macrophages | 28.51 | 20.16 | 8.35 | 3.05 |
| Kidney | 30.38 | 22.60 | 7.78 | 4.54 |
| Skeletal Muscle | 33.40 | 22.76 | 10.65 | 0.62 |
| Skin | 31.49 | 21.72 | 9.77 | 1.15 |
| Chondrocytes | 31.57 | 21.12 | 10.45 | 0.71 |
| Trachea | 31.85 | 22.03 | 9.82 | 1.11 |
| Fetal Lung | 29.20 | 23.75 | 5.45 | 22.83 |
| Lung | 30.97 | 21.01 | 9.96 | 1.01 |
| Lung COPD Control | 34.63 | 21.46 | 13.17 | 0.11 |
| Lung COPD | 32.57 | 21.55 | 11.02 | 0.48 |
| Lung Tumor Control | 32.78 | 20.29 | 12.49 | 0.17 |
| Lung | 30.87 | 19.82 | 11.05 | 0.47 |
| Tumor (adenocarcinoma) | | | | |
| Lung Tumor (carcinoma) | 29.76 | 20.69 | 9.07 | 1.86 |
| A549 cells | 31.21 | 21.15 | 10.06 | 0.94 |
| Breast | 28.92 | 23.23 | 5.70 | 19.29 |
| Breast Tumor Control | 30.72 | 22.01 | 8.71 | 2.39 |
| Breast | 30.57 | 21.33 | 9.24 | 1.66 |
| Tumor (adenocarcinoma) | | | | |
| Mammary Gland | 31.14 | 21.63 | 9.51 | 1.38 |
| Colon | 33.75 | 21.03 | 12.72 | 0.15 |
| Colon IBD Control | 33.12 | 20.13 | 12.99 | 0.12 |
| Colon IBD | 30.25 | 19.15 | 11.10 | 0.46 |
| Colon Tumor Control | 32.80 | 20.89 | 11.91 | 0.26 |
| Colon | 30.83 | 20.20 | 10.63 | 0.63 |
| Tumor (adenocarcinoma) | | | | |
| Esophagus | 32.49 | 23.20 | 9.29 | 1.60 |
| Stomach | 32.13 | 22.16 | 9.96 | 1.00 |
| Small Intestine | 32.84 | 22.34 | 10.50 | 0.69 |
| Pancreas | 33.67 | 24.81 | 8.86 | 2.15 |
| Ovary | 31.63 | 22.36 | 9.27 | 1.62 |
| Ovary Tumor Control | 31.59 | 21.71 | 9.88 | 1.06 |
| Ovary | 30.43 | 20.50 | 9.94 | 1.02 |
| Tumor (adenocarcinoma) | | | | |
| Ovary Tumor (carcinoma) | 31.36 | 20.21 | 11.15 | 0.44 |
| Prostate | 32.00 | 21.38 | 10.62 | 0.63 |
| Prostate Tumor Control | 32.00 | 21.95 | 10.04 | 0.95 |
| Prostate Tumor | 31.53 | 21.43 | 10.11 | 0.91 |
| (adenocarcinoma) | | | | |
| HeLa cells | 31.34 | 20.43 | 10.91 | 0.52 |
| Uterus | 29.07 | 21.57 | 7.49 | 5.55 |
| Placenta | 32.19 | 23.28 | 8.92 | 2.07 |
| Testis | 29.04 | 23.44 | 5.60 | 20.60 |
| Positive Control, HIPCO | 27.78 | 21.54 | 6.24 | 13.27 |

### REFERENCES

(1) Henrik Nielsen, Jacob Engelbrecht, Søren Brunak and Gunnar von Heijne “Identification of prokaryotic and eukaryotic signal peptides and prediction of their cleavage sites” Protein Engineering, 10:1-6, 1997.
(2) A. Krogh, B. Larsson, G. von Heijne and E. L. L. Sonnhammer. “Predicting transmembrane protein topology with a hidden Markov model: Application to complete genomes” Journal of Molecular Biology, 305(3):567-580, January 2001.
(3) Peter Duckert, Søren Brunak and Nikolaj Blom, “Prediction of proprotein convertase cleavage sites” Protein Engineering, Design and Selection: 17: 107-112, 2004.
(4) Strand, F. L. Neuropeptides: Regulators of physiological processes. (1999) MIT Press.
(5) Anderson, N. L. & Anderson, N. G. The human plasma proteome: history, character, and diagnostic prospects. Mol. Cell. Proteomics, 2002 November; 1(11): 845-67
(6) Mulder N J, Apweiler R, Attwood T K, Bairoch A, Bateman A, Binns D, Bradley P, Bork P, Bucher P, Cerutti L, Copley R, Courcelle E, Das U, Durbin R, Fleischmann W, Gough J, Haft D, Harte N, Hulo N, Kahn D, Kanapin A, Krestyaninova M, Lonsdale D, Lopez R, Letunic I, Madera M, Maslen J, McDowall J, Mitchell A, Nikolskaya A N, Orchard S, Pagni M, Ponting C P, Quevillon E, Selengut J, Sigrist C J, Silventoinen V, Studholme D J, Vaughan R, Wu C H. InterPro, progress and status in 2005. Nucleic Acids Res. 2005 Jan. 1; 33(Database issue):D201-5.

### DESCRIPTION OF THE FIGURES

FIG. 1: shows mRNA Expression Profile of AC105940 on ATAQ 1 for Seq Id no 1

FIG. 2: shows mRNA Expression Profile of AC005291 on ATAQ 1 for Seq Id no 3

FIG. 3: shows mRNA Expression Profile of AC090617 on ATAQ 1 for Seq Id no 4

FIG. 4: shows mRNA Expression Profile of AC114684 on ATAQ 1 for Seq Id no 5

FIG. 5: shows mRNA Expression Profile of AC063920 on ATAQ 1 for Seq Id no 7

FIG. 6: shows mRNA Expression Profile of AC074389 on ATAQ 1 for Seq Id no 8

FIG. 7: shows amino acid sequence list for Seq Id no 1

FIG. 8: shows amino acid sequence for Seq Id no 2

FIG. 9: shows amino acid sequence for Seq Id no 3

FIG. 10: shows amino acid sequence for Seq Id no 4

FIG. 11: shows amino acid sequence for Seq Id no 5

FIG. 12: shows amino acid sequence for Seq Id no 6

FIG. 13: shows amino acid sequence for Seq Id no 7

FIG. 14: shows amino acid sequence for Seq Id no 8

FIG. 15: shows nucleotide sequence for Seq Id no 9

FIG. 16: shows nucleotide sequence for Seq Id no 10

FIG. 17: shows nucleotide sequence for Seq Id no 11

FIG. 18: shows nucleotide sequence for Seq Id no 12

FIG. 19: shows nucleotide sequence for Seq Id no 13

FIG. 20: shows nucleotide sequence for Seq Id no 14

FIG. 21: shows nucleotide sequence for Seq Id no 15

FIG. 22: shows nucleotide sequence for Seq Id no 16

FIG. 23: shows nucleotide Probe sequence for Seq Id no 1

FIG. 24: shows nucleotide Forward primer sequence for Seq Id no 1

FIG. 25: shows nucleotide Reverse primer sequence for Seq Id no 1

FIG. 26: shows nucleotide Probe sequence for Seq Id no 2

FIG. 27: shows nucleotide Forward primer sequence for Seq Id no 2

FIG. 28: shows nucleotide Reverse primer sequence for Seq Id no 2

FIG. 29: shows nucleotide Probe sequence for Seq Id no 3

FIG. 30: shows nucleotide Forward primer sequence for Seq Id no 3

FIG. 31: shows nucleotide Reverse primer sequence for Seq Id no 3

FIG. 32: shows nucleotide Probe sequence for Seq Id no 4

FIG. 33: shows nucleotide Forward primer sequence for Seq Id no 4

FIG. 34: shows nucleotide Reverse primer sequence for Seq Id no 4

FIG. 35: shows nucleotide Probe sequence for Seq Id no 5

FIG. 36: shows nucleotide Forward primer sequence for Seq Id no 5

FIG. 37: shows nucleotide Reverse primer sequence for Seq Id no 5

FIG. 38: shows nucleotide Probe sequence for Seq Id no 6

FIG. 39: shows nucleotide Forward primer sequence for Seq Id no 6

FIG. 40: shows nucleotide Reverse primer sequence for Seq Id no 6

FIG. 41: shows nucleotide Probe sequence for Seq Id no 7

FIG. 42: shows nucleotide Forward primer sequence for Seq Id no 7

FIG. 43: shows nucleotide Reverse primer sequence for Seq Id no 7

FIG. 44: shows nucleotide Probe sequence for Seq Id no 8

FIG. 45: shows nucleotide Forward primer sequence for Seq Id no 8

FIG. 46: shows nucleotide Reverse primer sequence for Seq Id no 8

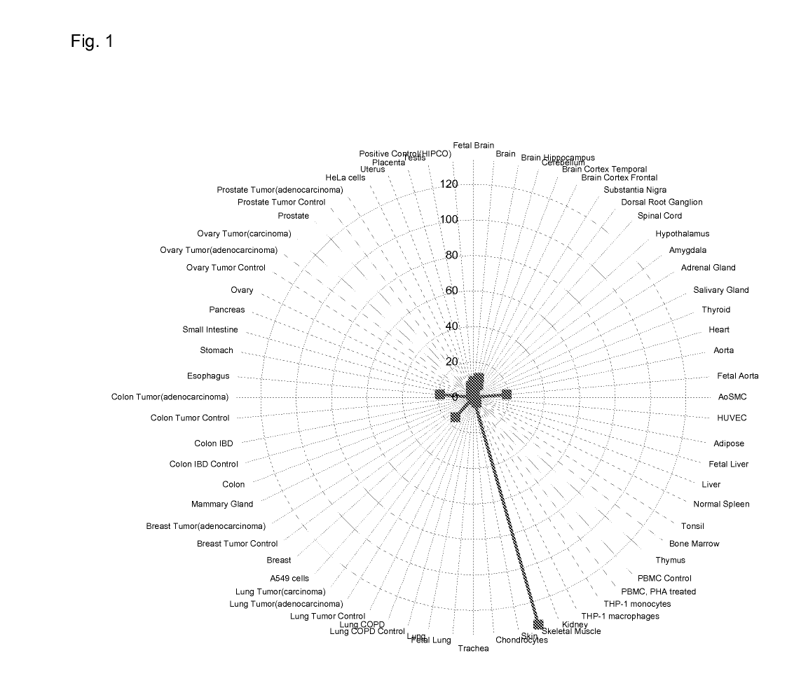

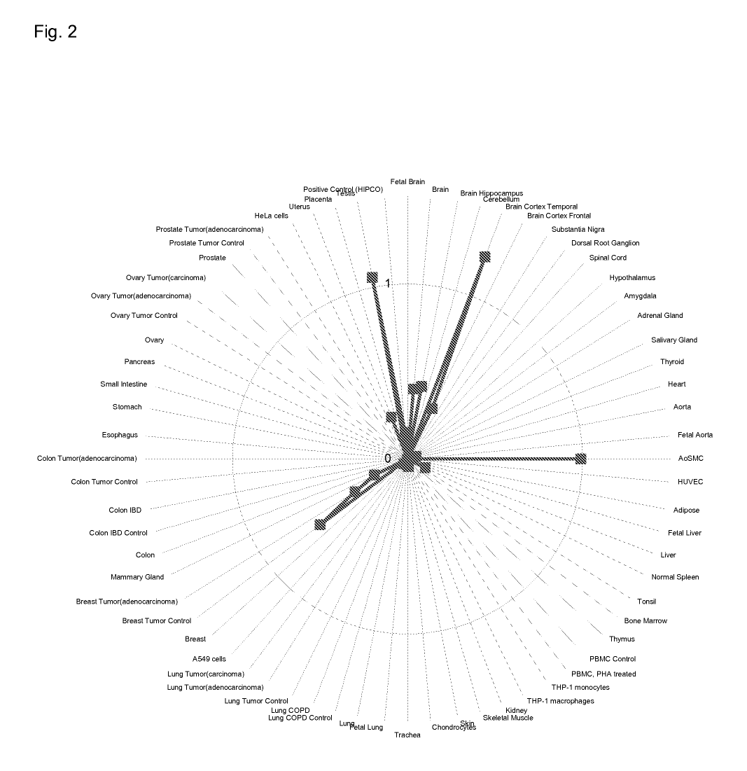

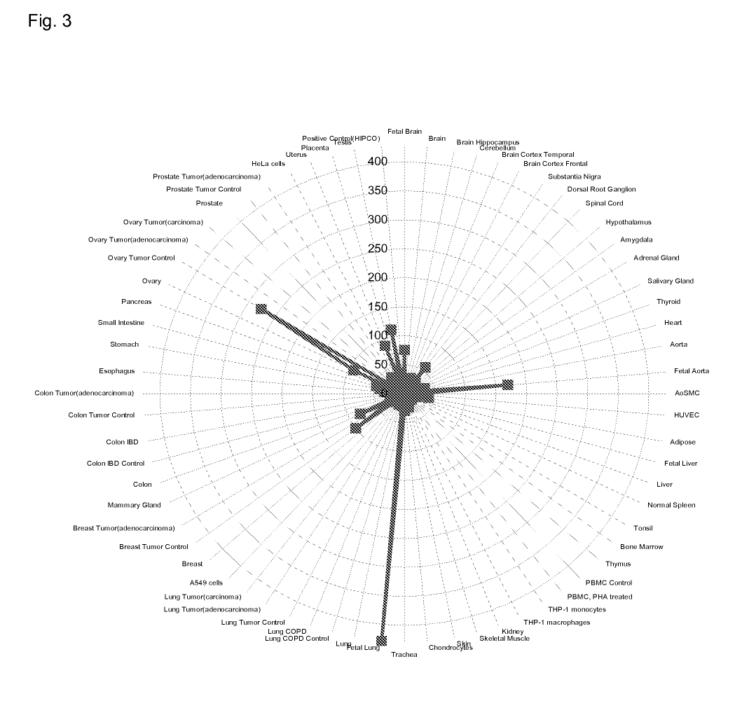

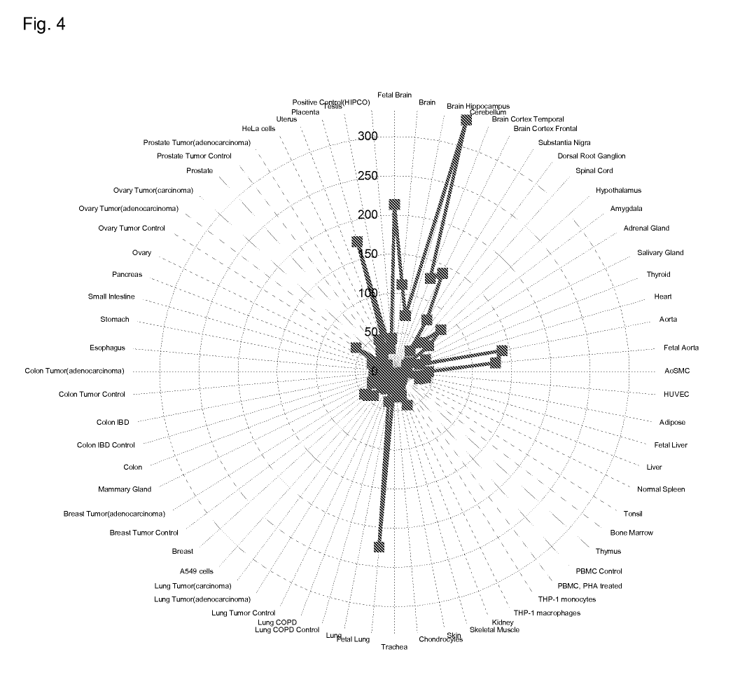

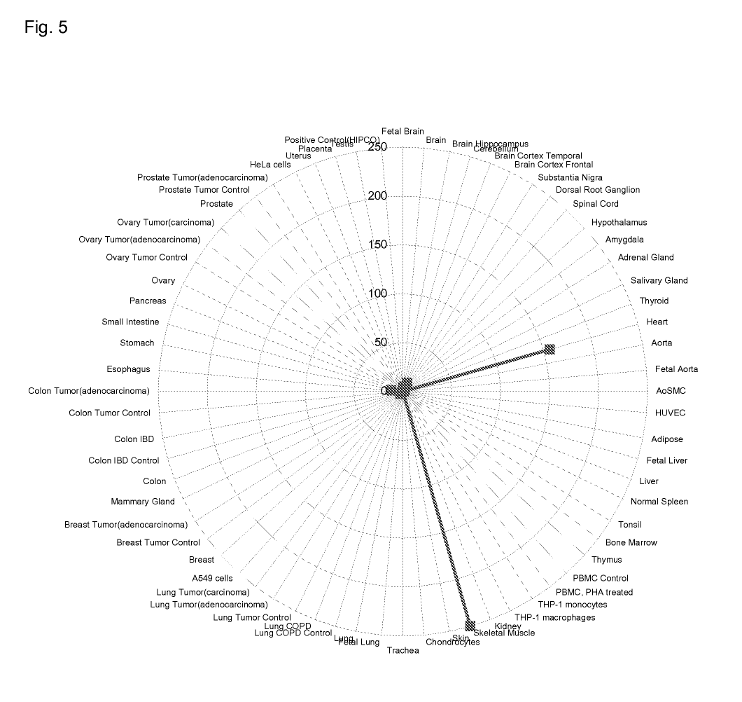

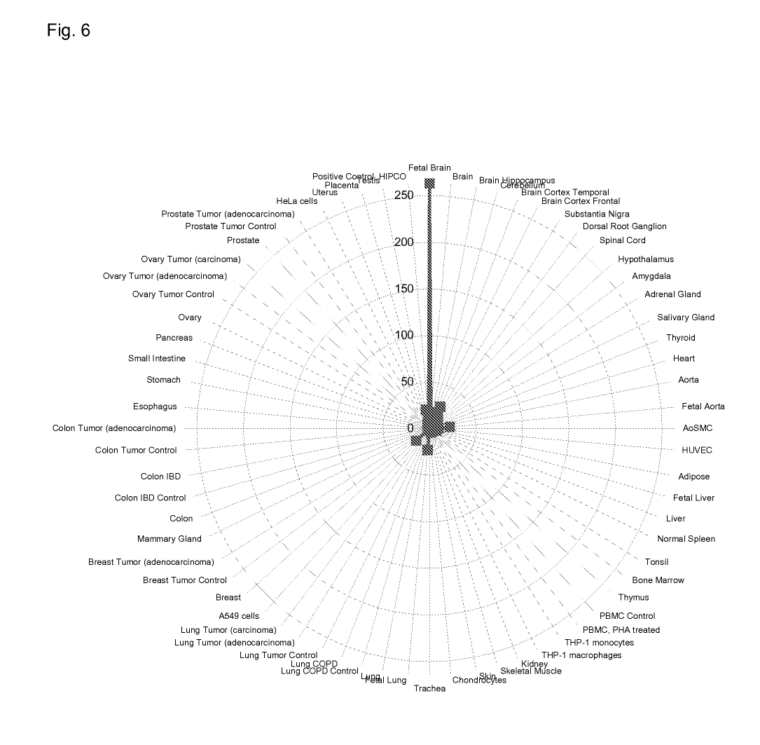

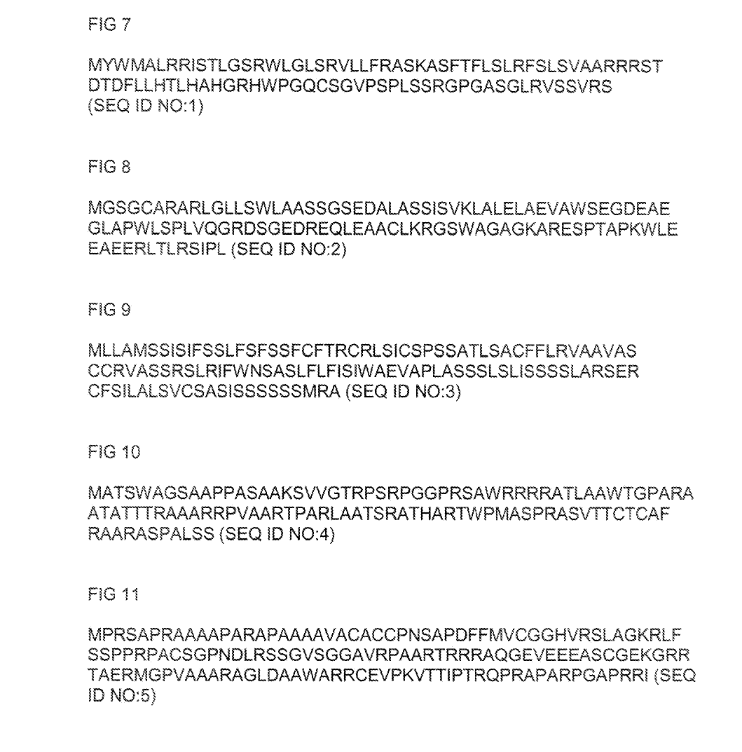

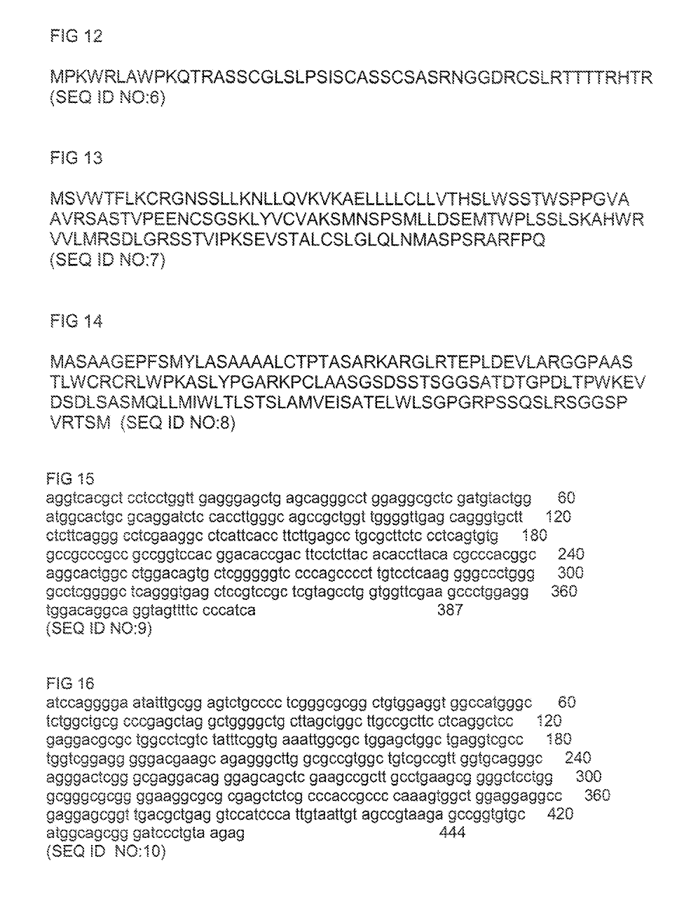

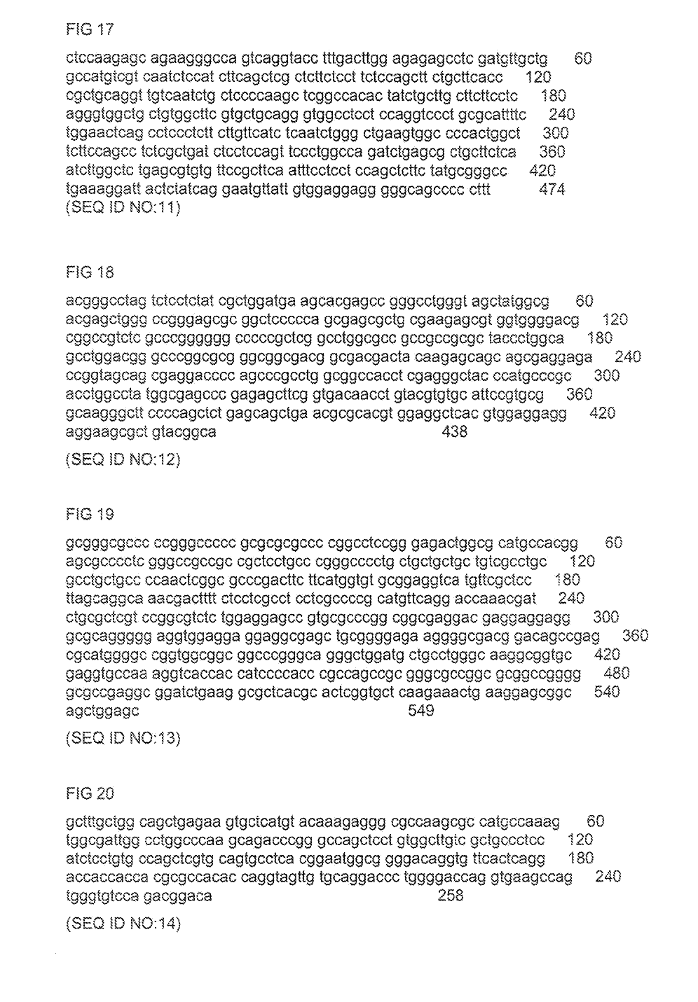

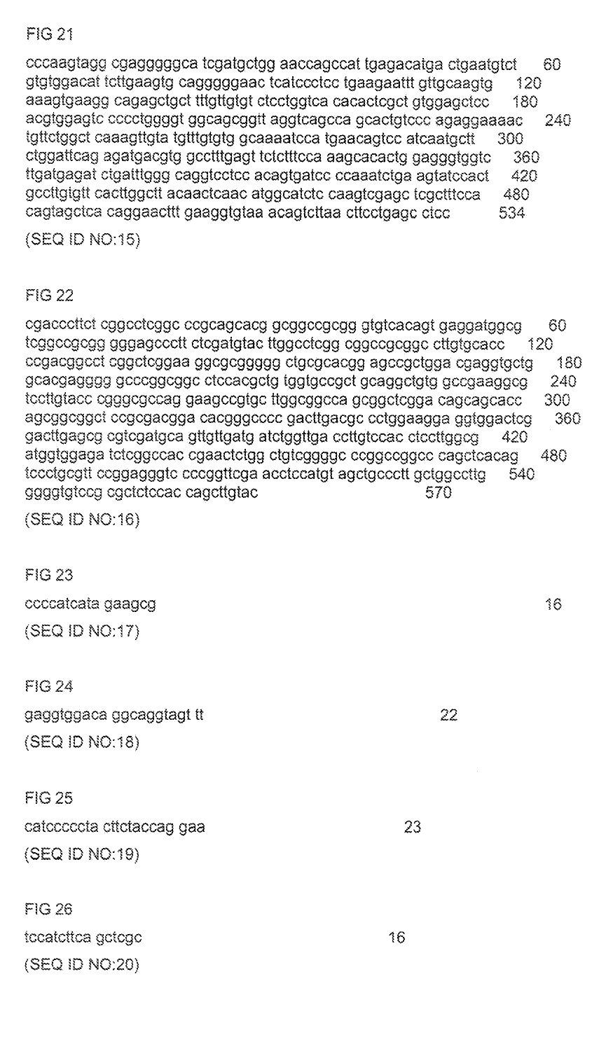

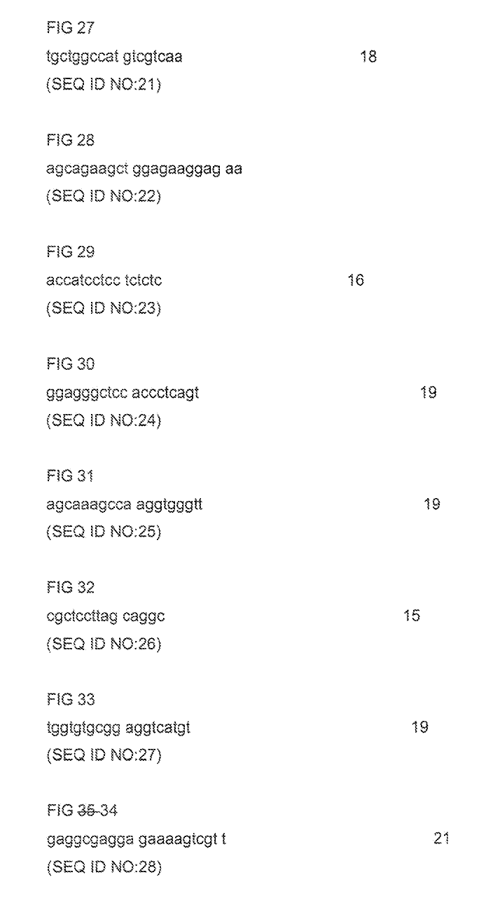

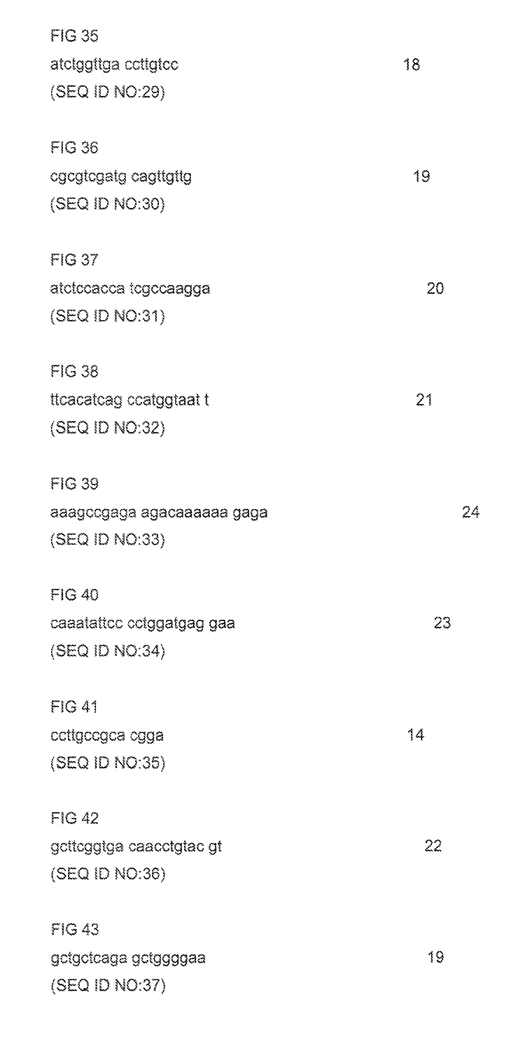

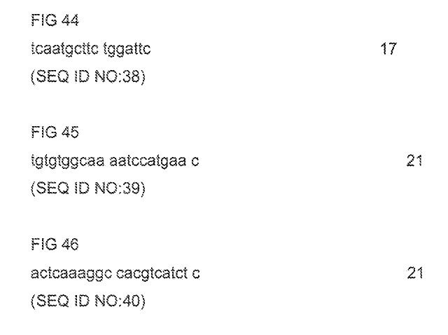

1. A polypeptide comprising any of the amino acid sequences of SEQ ID NOs:108 or an amino acid sequence derived there from by deletion, substitution or insertion of at least one amino acid residue, a precursor thereof, an amide or ester thereof, or a salt thereof.

2. The polypeptide of claim 1 consisting of the following amino acid sequence:
SEQ ID NO: 1
MYWMALRRISTLGSRWLGLSRVLLFRASKASFTFLSLRFSLSVAARRRST
DTDFLLHTLHAHGRHWPGQCSGVPSPLSSRGPGASGLRVSSVRS.

3. The polypeptide of claim 1 consisting of the following amino acid sequence:
(SEQ ID NO: 2)
MGSGCARARLGLLSWLAASSGSEDALASSISVKLALELAEVAWSEGDEAE
GLAPWLSPLVQGRDSGEDREQLEAACLKRGSWAGAGKARELSPTAPKWLE
EAEERLTLRSIPL.

4. The polypeptide of claim 1 consisting of the following amino acid sequence:
(SEQ ID NO: 3)
MLLAMSSISIFSSLFSFSSFCFTRCRLSICSPSSATLSACFFLRVAAVAS
CCRVASSRSLRIFWNSASLFLFISIWAEVAPLASSSLSLISSSSLARSER
CFSILALSVCSASISSSSSSMRA.

5. The polypeptide of claim 1 consisting of the following amino acid sequence:
(SEQ ID NO: 4)
MATSWAGSAAPPASAAKSVVGTRPSRPGGPRSAWRRRRATLAAWTGPARA
ATATTTRAAARRPVAARTPARLAATSRATHARTWPMASPRASVTTCTCAF
RAARASPALSS.

6. The polypeptide of claim 1 consisting of the following amino acid sequence:
(SEQ ID NO: 5)
MPRSAPRAAAAPARAPAAAAVACACCPNSAPDFFMVCGGHVRSLAGKRLF
SSPPRPACSGPNDLRSSGVSGGAVRPAARTRRRAQGEVEEEASCGEKGRR
TAERMGPVAAARAGLDAAWARRCEVPKVTTIPTRQPRAPARPGAPRRI.

7. The polypeptide of claim 1 consisting of the following amino acid sequence:
(SEQ ID NO: 6)
MPKWRLAWPKQTRASSCGLSLPSISCASSCSASRNGGDRCSLRTTTTRHT
R

8. The polypeptide of claim 1 consisting of the following amino acid sequence:
(SEQ ID NO: 7)
MSVWTFLKCRGNSSLLKNLLQVKVKAELLLLCLLVTHSLWSSTWSPPGVA
AVRSASTVPEENCSGSKLYVCVAKSMNSPSMLLDSEMTWPLSSLSKAHWR
VVLMRSDLGRSSTVIPKSEVSTALCSLGLQLNMASPSRARFPQ.

9. The polypeptide of claim 1 consisting of the following amino acid sequence:
(SEQ ID NO: 8)
MASAAGEPFSMYLASAAAALCTPTASARKARGLRTEPLDEVLARGGPAAS
TLWCRCRLWPKASLYPGARKPCLAASGSDSSTSGGSATDTGPDLTPWKEV
DSDLSASMQLLMIWLTLSTSLAMVELSATELWLSGPGRPSSQSLRSGGSP
VRTSM.

10. A DNA molecule comprising the nucleotide sequence of any of SEQ ID NOs:9-16 encoding the polypeptide of claim 1 comprising any of the amino acid sequences of SEQ ID NOs:1-8, its amide, its ester or a salt thereof.

11. The DNA molecule of claim 10 consisting of the nucleotide sequence of SEQ ID NO:9 that encodes a polypeptide consisting of the amino acid sequence of SEQ ID NO: 1.

12. The DNA molecule of claim 10 consisting of the nucleotide sequence of SEQ ID NO:10, encoding a polypeptide that consists of the amino acid sequence of SEQ ID NO:2.

13. The DNA molecule of claim 10 consisting of the nucleotide sequence of SEQ ID NO:11, encoding a polypeptide consisting of the amino acid sequence of SEQ ID NO:3.

14. The DNA molecule of claim 10 consisting of the nucleotide sequence of SEQ ID NO:12, encoding a polypeptide consisting of the amino acid sequence of SEQ ID NO:4.

15. The DNA molecule of claim 10 consisting of the nucleotide sequence of SEQ ID NO:13, encoding a polypeptide consisting of the amino acid sequence of SEQ ID NO:5.

16. The DNA molecule of claim 10 consisting of the nucleotide sequence of SEQ ID NO:14, encoding a polypeptide consisting of the amino acid sequence of SEQ ID NO:6.

17. The DNA molecule of claim 10 consisting of the nucleotide sequence of SEQ ID NO:15, encoding a polypeptide consisting of the amino acid sequence of SEQ ID NO:7.

18. The DNA molecule of claim 10 consisting of the nucleotide sequence of SEQ ID NO:16, encoding a polypeptide consisting of the amino acid sequence of SEQ ID NO:8.

19. A recombinant vector comprising the DNA molecule of claim 10.

20. A method for manufacturing the polypeptide of claim 1, comprising the steps of:
i. providing the amino acids;
ii. synthesizing the polypeptide with the amino acids of step i by solid phase or liquid phase synthesis
iii. extracting the polypeptide; and
iv. purifying the polypeptide

21. A method of producing the polypeptide of claim 1, comprising subjecting an amino terminus-constituting amino acid or peptide and a carboxyl terminus-constituting amino acid or peptide to condensation.

22. A pharmaceutical composition comprising as the active agent the polypeptide of claim 1, and a pharmaceutically acceptable carrier thereof.

23. A method for changing physiological factors that increase the risk of arteriorsclerosis, inflammation or uncontrolled cell division in a subject comprising administering an effective amount of the pharmaceutical composition of claim 22.

24. An antibody to the polypeptide of claim 1.

25. A cell transformed or transfected with the recombinant vector of claim 19.

26. The method of claim 21, further comprising the step of forming intramolecular disulfide bonds.

27. A DNA molecule that encodes the polypeptide of claim 1.

28. An expression vector comprising the DNA molecule of 27 operatively associated with a promoter.

29. A cell transformed or transfected with the expression vector of claim 28.

30. A method of expressing a polypeptide comprising any of the amino acid sequences of SEQ ID NOs:1-8, comprising culturing the cell of claim 29 in an appropriate cell culture medium under conditions that provide for expression of polypeptide by the cell.

31. The method of claim 30, further comprising the step of purifying the polypeptide.


SEQUENCE LISTING (40 sequences)

SEQ ID NO: 1 - protein, 94 aa, Homo sapiens
MYWMALRRISTLGSRWLGLSRVLLFRASKASFTFLSLRFSLSVAARRRSTDTDFLLHTLHAHGRHWPGQCSGVPSPLSSRGPGASGLRVSSVRS

SEQ ID NO: 2 - protein, 113 aa, Homo sapiens [sequence omitted]

SEQ ID NO: 3 - protein, 123 aa, Homo sapiens
MLLAMSSISIFSSLFSFSSFCFTRCRLSICSPSSATLSACFFLRVAAVASCCRVASSRSLRIFWNSASLFLFISIWAEVAPLASSSLSLISSSSLARSERCFSILALSVCSASISSSSSSMRA

SEQ ID NO: 4 - protein, 111 aa, Homo sapiens
MATSWAGSAAPPASAAKSVVGTRPSRPGGPRSAWRRRRATLAAWTGPARAATATTTRAAARRPVAARTPARLAATSRATHARTWPMASPRASVTTCTCAFRAARASPALSS

SEQ ID NO: 5 - protein, 148 aa, Homo sapiens
MPRSAPRAAAAPARAPAAAAVACACCPNSAPDFFMVCGGHVRSLAGKRLFSSPPRPACSGPNDLRSSGVSGGAVRPAARTRRRAQGEVEEEASCGEKGRRTAERMGPVAAARAGLDAAWARRCEVPKVTTIPTRQPRAPARPGAPRRI

SEQ ID NO: 6 - protein, 51 aa, Homo sapiens
MPKWRLAWPKQTRASSCGLSLPSISCASSCSASRNGGDRCSLRTTTTRHTR

SEQ ID NO: 7 - protein, 143 aa, Homo sapiens
MSVWTFLKCRGNSSLLKNLLQVKVKAELLLLCLLVTHSLWSSTWSPPGVAAVRSASTVPEENCSGSKLYVCVAKSMNSPSMLLDSEMTWPLSSLSKAHWRVVLMRSDLGRSSTVIPKSEVSTALCSLGLQLNMASPSRARFPQ

SEQ ID NO: 8 - protein, 155 aa, Homo sapiens
MASAAGEPFSMYLASAAAALCTPTASARKARGLRTEPLDEVLARGGPAASTLWCRCRLWPKASLYPGARKPCLAASGSDSSTSGGSATDTGPDLTPWKEVDSDLSASMQLLMIWLTLSTSLAMVEISATELWLSGPGRPSSQSLRSGGSPVRTSM

SEQ ID NO: 9 - DNA, 387 bp, Homo sapiens
aggtcacgctcctcctggttgagggagctgagcagggcctggaggcgctcgatgtactggatggcactgcgcaggatctccaccttgggcagccgctggttggggttgagcagggtgcttctcttcagggcctcgaaggcctcattcaccttcttgagcctgcgcttctccctcagtgtggccgcccgccgccggtccacggacaccgacttcctcttacacaccttacacgcccacggcaggcactggcctggacagtgctcgggggtccccagccccttgtcctcaaggggccctggggcctcggggctcagggtgagctccgtccgctcgtagcctggtggttcgaagccctggaggtggacaggcaggtagttttccccatca

SEQ ID NO: 10 - DNA, 444 bp, Homo sapiens
atccaggggaatatttgcggagtctgcccctcgggcgcggctgtggaggtggccatgggctctggctgcgcccgagctaggctggggctgcttagctggcttgccgcttcctcaggctccgaggacgcgctggcctcgtctatttcggtgaaattggcgctggagctggctgaggtcgcctggtcggagggggacgaagcagagggcttggcgccgtggctgtcgccgttggtgcagggcagggactcgggcgaggacagggagcagctcgaagccgcttgcctgaagcggggctcctgggcgggcgcggggaaggcgcgcgagctctcgcccaccgccccaaagtggctggaggaggccgaggagcggttgacgctgaggtccatcccattgtaattgtagccgtaagagccggtgtgcatggcagcgggatccctgtaagag

SEQ ID NO: 11 - DNA, 474 bp, Homo sapiens
ctccaagagcagaagggccagtcaggtacctttgacttggagagagcctcgatgttgctggccatgtcgtcaatctccatcttcagctcgctcttctccttctccagcttctgcttcacccgctgcaggttgtcaatctgctccccaagctcggccacactatctgcttgcttcttcctcagggtggctgctgtggcttcgtgctgcagggtggcctcctccaggtccctgcgcattttctggaactcagcctccctcttcttgttcatctcaatctgggctgaagtggccccactggcttcttccagcctctcgctgatctcctccagttccctggccagatctgagcgctgcttctcaatcttggctctgagcgtgtgttccgcttcaatttcctcctccagctcttctatgcgggcctgaaaggattactctatcaggaatgttattgtggaggagggggcagcccccttt

SEQ ID NO: 12 - DNA, 438 bp, Homo sapiens
acgggcctagtctcctctatcgctggatgaagcacgagccgggcctgggtagctatggcgacgagctgggccgggagcgcggctcccccagcgagcgctgcgaagagcgtggtggggacgcggccgtctcgcccggggggcccccgctcggcctggcgccgccgccgcgctaccctggcagcctggacgggcccggcgcgggcggcgacggcgacgactacaagagcagcagcgaggagaccggtagcagcgaggaccccagcccgcctggcggccacctcgagggctacccatgcccgcacctggcctatggcgagcccgagagcttcggtgacaacctgtacgtgtgcattccgtgcggcaagggcttccccagctctgagcagctgaacgcgcacgtggaggctcacgtggaggaggaggaagcgctgtacggca

SEQ ID NO: 13 - DNA, 549 bp, Homo sapiens
gcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagc

SEQ ID NO: 14 - DNA, 258 bp, Homo sapiens
gctttgctggcagctgagaagtgctcatgtacaaagagggcgccaagcgccatgccaaagtggcgattggcctggcccaagcagacccgggccagctcctgtggcttgtcgctgccctccatctcctgtgccagctcgtgcagtgcctcacggaatggcggggacaggtgttcactcaggaccaccaccacgcgccacaccaggtagttgtgcaggaccctggggaccaggtgaagccagtgggtgtccagacggaca

SEQ ID NO: 15 - DNA, 534 bp, Homo sapiens
cccaagtaggcgagggggcatcgatgctggaaccagccattgagacatgactgaatgtctgtgtggacattcttgaagtgcagggggaactcatccctcctgaagaatttgttgcaagtgaaagtgaaggcagagctgcttttgttgtgtctcctggtcacacactcgctgtggagctccacgtggagtccccctggggtggcagcggttaggtcagccagcactgtcccagaggaaaactgttctggctcaaagttgtatgtttgtgtggcaaaatccatgaacagtccatcaatgcttctggattcagagatgacgtggcctttgagttctctttccaaagcacactggagggtggtcttgatgagatctgatttgggcaggtcctccacagtgatccccaaatctgaagtatccactgccttgtgttcacttggcttacaactcaacatggcatctccaagtcgagctcgctttccacagtagctcacaggaactttgaaggtgtaaacagtcttaacttcctgagcctcc

SEQ ID NO: 16 - DNA, 570 bp, Homo sapiens
cgacccttctcggcctcggcccgcagcacggcggccgcgggtgtcacagtgaggatggcgtcggccgcgggggagcccttctcgatgtacttggcctcggcggccgcggccttgtgcaccccgacggcctcggctcggaaggcgcgggggctgcgcacggagccgctggacgaggtgctggcacgagggggcccggcggcctccacgctgtggtgccgctgcaggctgtggccgaaggcgtccttgtacccgggcgccaggaagccgtgcttggcggccagcggctcggacagcagcaccagcggcggctccgcgacggacacgggccccgacttgacgccctggaaggaggtggactcggacttgagcgcgtcgatgcagttgttgatgatctggttgaccttgtccacctccttggcgatggtggagatctcggccaccgaactctggctgtcggggcccggccggcccagctcacagtccctgcgttccggagggtccccggttcgaacctccatgtagctgcccttgctggccttgggggtgtccgcgctctccaccagcttgtac

SEQ ID NO: 17 - DNA, 16 bp, Artificial
  probe sequence
ccccatcatagaagcg

SEQ ID NO: 18 - DNA, 22 bp, Artificial
  Forward primer
gaggtggacaggcaggtagttt

SEQ ID NO: 19 - DNA, 23 bp, Artificial
  Reverse primer
catccccctacttctaccaggaa

SEQ ID NO: 20 - DNA, 16 bp, Artificial
  Probe sequence
tccatcttcagctcgc

SEQ ID NO: 21 - DNA, 18 bp, Artificial
  Forward primer
tgctggccatgtcgtcaa

SEQ ID NO: 22 - DNA, 22 bp, Artificial
  Reverse primer
agcagaagctggagaaggagaa

SEQ ID NO: 23 - DNA, 16 bp, Artificial
  Probe sequence
accatcctcctctctc

SEQ ID NO: 24 - DNA, 19 bp, Artificial
  Forward primer
ggagggctccaccctcagt

SEQ ID NO: 25 - DNA, 19 bp, Artificial
  Reverse primer
agcaaagccaaggtgggtt

SEQ ID NO: 26 - DNA, 15 bp, Artificial
  Probe sequence
cgctccttagcaggc

SEQ ID NO: 27 - DNA, 19 bp, Artificial
  Forward primer
tggtgtgcggaggtcatgt

SEQ ID NO: 28 - DNA, 21 bp, Artificial
  Reverse primer
gaggcgaggagaaaagtcgtt

SEQ ID NO: 29 - DNA, 18 bp, Artificial
  Probe sequence
atctggttgaccttgtcc

SEQ ID NO: 30 - DNA, 19 bp, Artificial
  Forward primer
cgcgtcgatgcagttgttg

SEQ ID NO: 31 - DNA, 20 bp, Artificial
  Reverse primer
atctccaccatcgccaagga

SEQ ID NO: 32 - DNA, 21 bp, Artificial
  Probe sequence
ttcacatcagccatggtaatt

SEQ ID NO: 33 - DNA, 24 bp, Artificial
  Forward primer
aaagccgagaagacaaaaaagaga

SEQ ID NO: 34 - DNA, 23 bp, Artificial
  Reverse primer
caaatattcccctggatgaggaa

SEQ ID NO: 35 - DNA, 14 bp, Artificial
  Probe sequence
ccttgccgcacgga

SEQ ID NO: 36 - DNA, 22 bp, Artificial
  Forward primer
gcttcggtgacaacctgtacgt

SEQ ID NO: 37 - DNA, 19 bp, Artificial
  Reverse primer
gctgctcagagctggggaa

SEQ ID NO: 38 - DNA, 17 bp, Artificial
  Probe Sequence
tcaatgcttctggattc

SEQ ID NO: 39 - DNA, 21 bp, Artificial
  Forward primer
tgtgtggcaaaatccatgaac

SEQ ID NO: 40 - DNA, 21 bp, Artificial
  Reverse primer
actcaaaggccacgtcatctc